(19) United States
(12) Patent Application Publication
(10) Pub. No.: US 2008/0033296 A1
(43) Pub. Date: Feb. 7, 2008

(54) METHODS FOR DELIVERING MATERIALS INTO BIOLOGICAL SYSTEMS USING SONIC ENERGY

(75) Inventor: Sharon ISERN, Estero, FL (US)

Correspondence Address: HAHN LOESER & PARKS, LLP, One GOJO Plaza, Suite 300, AKRON, OH, 44311-1076, US

(73) Assignee: FLORIDA GULF COAST UNIVERSITY, Fort Myers, FL (US)

(21) Appl. No.: 11/675,349

(22) Filed: Feb. 15, 2007

Related U.S. Application Data
(60) Provisional application No. 60/774,429, filed on Feb. 17, 2006.

(51) Int. Cl. A61B 8/00

(52) U.S. Cl. 600/439

(57) ABSTRACT

Methods to facilitate delivering of materials into biological systems using sonic energy. The processes include a method of controllably disrupting cell membranes and/or cell walls in biological cells using sonic energy, methods of introducing materials into biological cells using sonic energy, and methods of correlating sonic energy exposure to material transport efficiency into biological cells.

### CROSS-REFERENCE TO RELATED APPLICATIONS/INCORPORATION BY REFERENCE

This U.S. patent application claims priority to and the benefit of Provisional U.S. Patent Application Ser. No. 60/774,429 filed on Feb. 17, 2006, which is incorporated by reference herein in its entirety. Furthermore, U.S. Pat. No. 6,719,449 issued Apr. 13, 2004 and U.S. Patent Application Publication Number 2004/0264293 published Dec. 30, 2004 are also incorporated herein by reference in their entirety.

### GOVERNMENT SUPPORT

The U.S. Government has a paid-up license in this invention and the right in limited circumstances to require the patent owner to license others on reasonable terms as provided for by the terms of contract/grant no. 0521917 awarded by the National Science Foundation and grant no. 2004-LC-CX-K048 awarded by the Department of Justice and contract no. N00173-06-1-G901 awarded by the Department of Defense.

### TECHNICAL FIELD

Certain embodiments of the present invention relate to delivering materials into biological systems. More particularly, certain embodiments of the present invention relate to delivering materials into biological cells using sonic energy.

### BACKGROUND

Introduction of biological and non-biological materials into cells, tissues, and whole organisms has many useful applications in a wide variety of fields. Such materials include but are not limited to chemicals, inorganic and organic molecules, monomers including saccharides, nucleic acids, amino acids, polymers including polysaccharides, nucleic acid chains, and proteins, viruses, plasmids, and vectors.

Current methods for introducing materials into cells and tissues include chemical and lipid-based delivery methods, microinjection, electroporation, particle bombardment, molecular conjugates, and viral vectors. No one single method may efficiently be used to deliver every type of material into every target cell or tissue. The usefulness of existing delivery methods is limited by factors including tissue tropism, toxicity, viability of target cells and tissues and efficiency.

In general, existing delivery methods have been developed for a selected number of biological systems and, in particular, mammalian and bacterial systems. Methods of efficiently introducing materials into non-vertebrate plant or animal systems are still needed and would facilitate and advance scientific discovery in many fields where delivery methods are inefficient or lacking.

Further limitations and disadvantages of conventional, traditional, and proposed approaches will become apparent to one of skill in the art, through comparison of such systems and methods with embodiments of the present invention as set forth in the remainder of the present application with reference to the drawings.

### SUMMARY OF THE INVENTION

The following definitions apply herein:

The term “sonic energy” as used herein is intended to encompass such terms as acoustic energy, acoustic waves, acoustic pulses, ultrasonic energy, ultrasonic waves, ultrasound, shock waves, sound energy, sound waves, sonic pulses, pulses, waves, or any other grammatical form of these terms, as well as any other type of energy that has similar characteristics to acoustic energy.

The term “focal zone” or “focal point” as used herein means an area where sonic energy converges and/or impinges on a target, although that area of convergence is not necessarily a single focused point.

The terms “microplate,” “microtiter plate,” “microwell plate,” and other grammatical forms of these terms as used herein may mean a plate that includes one or more wells into which samples may be deposited.

The term “nonlinear sonics” as used herein may mean lack of proportionality between input and output. For example, water becomes nonlinear at high intensities, and in a converging sonic field, the waves become more disturbed as the intensity increases toward the focal point. Nonlinear sonic properties of tissue may be useful in diagnostic and therapeutic applications.

The term “sonic streaming” as used herein means generation of fluid flow by sonic waves. The effect may be non-linear. Bulk fluid flow of a liquid in the direction of the sound field may be created as a result of momentum absorbed from the sonic field.

The term “sonic microstreaming” as used herein means time-independent circulation that occurs only in a small region of the fluid around a source or obstacle for example, an acoustically driven bubble in a sound field.

The term “sonic absorption” as used herein refers to a characteristic of a material relating to the material's ability to convert sonic energy into thermal energy.

The term “sonic impedance” as used herein means a ratio of sound pressure on a surface to sound flux through the surface, the ratio having a reactance and a resistance component.

The term “sonic lens” as used herein means a system or device for spreading or converging sounds waves.

The term “sonic scattering” means irregular and multi-directional reflection and diffraction of sound waves produced by multiple reflecting surfaces, the dimensions of which are small compared to the wavelength, or by certain discontinuities in the medium through which the wave is propagated.

The term “cavitation” as used herein means the nucleation, expansion and decay or collapse of a vacuum space (cavity) or gas/vapor space (bubble) in a fluid as a result of an acoustic pressure field.

The term “bubble” as used herein means a gas body or cavity in a fluid or at a fluid/solid interface having in its interior a vacuum, or a gas or mixture of gasses.

The term “couplant” as used herein means any single material or plurality of materials in a sonic path for coupling sonic energy from a source location to another location. A couplant may be a portion of a microdevice, such as a wall of a microdevice, used to couple sonic energy from a sonic source to an internal chamber of the microdevice.

The term “non-contact” as used herein refers to a sonic source not being in mechanical contact with a fluid to be controlled.

The term “active site” as used herein means location of a receptor or sensor of any kind, such as, nucleic acid, nucleic acid probe, protein, antibody, small molecule, tissue sample and nonbiological material.

The term “biocompatible composition” as used herein means that the composition in question, upon implantation in a subject does not elicit a detrimental response sufficient to result in the rejection of the composition or to render it inoperable, for example through degradation. To determine whether any subject composition is biocompatible, it may be necessary to conduct a toxicity analysis. Such assays are well known in the art. One non-limiting example of such an assay for analyzing a composition of various embodiments would be performed with live carcinoma cells, such as GT3TKB tumor cells, in the following manner: various amounts of subject compositions are placed in 96-well tissue culture plates and seeded with human gastric carcinoma cells (GT3TKB) at 104/well density. The degraded products are incubated with the GT3TKB cells for 48 hours. The results of the assay may be plotted as % relative growth versus amount of compositions in the tissue-culture well. In addition, compositions of various embodiments may also be evaluated by well-known in vivo tests, such as subcutaneous implantations in rats to confirm that they do not cause significant levels of irritation or inflammation at the subcutaneous implantation sites.

The term “treating” is art recognized and includes preventing a disease, disorder or condition from occurring in a patient which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected. Treating includes, without limitation, use of the subject compositions with a diagnostic for diagnostic purposes as well as a targeting moiety or an antigen.

The term “active agent” includes without limitation, therapeutic agents, diagnostics, targeting moieties, and antigens.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

A “diagnostic” or “diagnostic agent” is any chemical moiety that may be used for diagnosis. For example, diagnostic agents include imaging agents containing radioisotopes such as indium or technetium; contrasting agents containing iodine or gadolinium; enzymes such as horse radish peroxidase, GFP, alkaline phosphatase, or β-galactosidase; fluorescent substances such as europium derivatives; luminescent substances such as N-methylacrydium derivatives or the like.

“Diagnosis” is intended to encompass diagnostic, prognostic, and screening methods.

The term “targeting moiety” refers to any molecular structure which assists the construct in localizing to a particular target area, entering a target cell(s), and/or binding to a target receptor. For example, lipids (including cationic, neutral, and steroidal lipids, virosomes, and liposomes), antibodies, lectins, ligands, sugars, steroids, hormones, nutrients, and proteins may serve as targeting moieties.

A “target” shall mean a site to which targeted constructs bind. A target may be either in vivo or in vitro. In certain embodiments, a target may be a tumor (e.g., tumors of the brain, lung (small cell and non-small cell), ovary, prostate, breast and colon as well as other carcinomas and sarcomas). In other embodiments, a target may be a site of infection (e.g., by bacteria, viruses (e.g., HIV, herpes, hepatitis) and pathogenic fungi (Candida sp.). In still other embodiments, a target may refer to a molecular structure to which a targeting moiety binds, such as a hapten, epitope, receptor, dsDNA fragment, carbohydrate or enzyme. Additionally, a target may be a type of tissue, e.g., neuronal tissue, intestinal tissue, pancreatic tissue etc.

The term “antigen” refers to any molecule or compound that specifically binds to an antigen binding site.

The term “antigen binding site” refers to a region of an antibody construct that specifically binds an epitope on an antigen.

The term “antibody” is art-recognized and intended to include whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. Embodiments may include polyclonal, monoclonal or other purified preparations of antibodies and recombinant antibodies.

The phrases “systemic administration,” “administered systemically,” “peripheral administration” and “administered peripherally” are art-recognized, and include the administration of a subject composition or other material at a site remote from the disease being treated. Administration of a subject composition directly into, onto or in the vicinity of a lesion of the disease being treated, even if the composition is subsequently distributed systemically, may be termed “local” or “topical” or “regional” administration, other than directly into the central nervous system, e.g., by subcutaneous administration, such that it enters the patient's system and, thus, is subject to metabolism and other like processes.

The phrase “therapeutically effective amount” is an art-recognized term. In certain embodiments, the term refers to an amount of the therapeutic agent that, when bridged through a metal ion to a carrier used in embodiments herein, produces some desired effect at a reasonable benefit/risk ratio applicable to any medical treatment. In certain embodiments, the term refers to that amount necessary or sufficient to eliminate, reduce or maintain (e.g., prevent the spread of) a tumor or other target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, the term refers to that amount necessary or sufficient for a use of the subject compositions described herein.

The term “naturally-occurring”, as applied to an object, refers to the fact that an object may be found in nature. For example, a carrier that may be isolated from a source in nature and which has not been intentionally modified by man in the laboratory is naturally-occurring.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in all animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which may readily be determined by one of ordinary skill in the art. For example, certain compounds used in certain embodiments, such as the subject coordination complex, may be administered in a sufficient amount to produce a reasonable benefit/risk ratio applicable to such treatment.

The terms “combinatorial library” or “library” are art-recognized and refer to a plurality of compounds, which may be termed “members,” synthesized or otherwise prepared from one or more starting materials by employing either the same or different reactants or reaction conditions at each reaction in the library. There are a number of other terms of relevance to combinatorial libraries (as well as other technologies). The term “identifier tag” is art-recognized and refers to a means for recording a step in a series of reactions used in the synthesis of a chemical library. The term “immobilized” is art-recognized and, when used with respect to a species, refers to a condition in which the species is attached to a surface with an attractive force stronger than attractive forces that are present in the intended environment of use of the surface, and that act on the species. The term “solid support” is art-recognized and refers to a material which is an insoluble matrix, and may (optionally) have a rigid or semi-rigid surface. The term “linker” is art-recognized and refers to a molecule or group of molecules connecting a support, including a solid support or polymeric support, and a combinatorial library member. The term “polymeric support” is art-recognized and refers to a soluble or insoluble polymer to which a chemical moiety may be covalently bonded by reaction with a functional group of the polymeric support. The term “functional group of a polymeric support” is art-recognized and refers to a chemical moiety of a polymeric support that may react with an chemical moiety to form a polymer-supported amino ester.

The term “synthetic” is art-recognized and refers to production by in vitro chemical or enzymatic synthesis.

The term “ED₅₀” is art-recognized and refers to the dose of a drug or other compound or coordination complex which produces 50% of its maximum response or effect, or alternatively, the dose which produces a pre-determined response in 50% of test subjects or preparations.

The term “LD₅₀” is art-recognized and refers to the dose of a drug or other compound or coordination complex which is lethal in 50% of test subjects.

The term “therapeutic index” is art-recognized and refers to the therapeutic index of a drug or other compound or coordination complex defined as LD₅₀/ED₅₀.

The term “agonist” is art-recognized and refers to a compound or coordination complex that mimics the action of natural transmitter or, when the natural transmitter is not known, causes changes at the receptor complex in the absence of other receptor ligands.

The term “antagonist” is art-recognized and refers to a compound or coordination complex that binds to a receptor site, but does not cause any physiological changes unless another receptor ligand is present.

The term “competitive antagonist” is art-recognized and refers to a compound or coordination complex that binds to a receptor site; its effects may be overcome by increased concentration of the agonist.

The term “partial agonist” is art-recognized and refers to a compound or coordination complex that binds to a receptor site but does not produce the maximal effect regardless of its concentration.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes herein, the heteroatoms such as nitrogen may have hydrogen substituents and/or any permissible substituents of organic compounds described herein which satisfy the valences of the heteroatoms, Embodiments are not intended to be limited in any manner by the permissible substituents of organic compounds.

For purposes herein, the chemical elements are identified in accordance with the Periodic Table of the Elements, CAS version, Handbook of Chemistry and Physics, 67th Ed., 1986-87, inside cover. Also for purposes herein, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “amino acid” is art-recognized and refers to all compounds, whether natural or synthetic, which include both an amino functionality and an acid functionality, including amino acid analogs and derivatives.

The terms “amino acid residue” and “peptide residue” are art-recognized and refer to an amino acid or peptide molecule without the —OH of its carboxyl group.

The term “amino acid residue” further includes analogs, derivatives and congeners of any specific amino acid referred to herein, as well as C-terminal or N-terminal protected amino acid derivatives (e.g. modified with an N-terminal or C-terminal protecting group).

The names of the natural amino acids are abbreviated herein in accordance with the recommendations of IUPAC-IUB.

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman et al. Accounts of Chem. Res. 12:423 (1979).

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds used in various embodiments may be formed by utilizing an appropriate amino acid mimetic moiety to link the two adjacent portions of the sequences depicted above utilizing a reversed peptide (reversed amide) bond.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is usually (D), and the configuration of the non-reversed portion is usually (L). Opposite or mixed configurations are acceptable when appropriate to optimize a binding activity.

The term “nucleic acid” is art-recognized and refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “polymer” is art-recognized and refers to any of numerous compounds of usually high molecular weight and consisting of up to millions of repeated linked units, each a relatively light and simple molecule. The polymer may be natural, such as cellulose or DNA, or synthetic, such as nylon or polyethylene.

The term “polysaccharide” is art-recognized and refers to any of a group of carbohydrates composed of long chains of simple sugars; e.g., starch, cellulose, insulin, or glycogen.

The term “nucleic acid chains” is art recognized and refers to any polyribonucleotide or polydeoxyribonucleotide, that may be unmodified RNA or DNA or modified RNA or DNA. “Nucleic acid chains” include, without limitation, single- and double-stranded DNA, DNA that is a mixture of single- and double-stranded regions or single-, double- and triple-stranded regions, single- and double-stranded RNA, and RNA that is a mixture of single- and double-stranded regions, hybrid molecules comprising DNA and RNA that may be single-stranded or, more typically, double-stranded, or triple-stranded regions, or a mixture of single- and double-stranded regions. In addition, “nucleic acid chains” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “nucleic acid chains” also includes DNAs or RNAs as described above that comprise one or more modified bases. Thus, DNAs or RNAs with backbones modified for stability or for other reasons are “nucleic acid chains” as that term is intended herein. Moreover, DNAs or RNAs comprising unusual bases, such as inosine, or modified bases, such as tritylated bases, to name just two examples, are nucleic acid chains as the term is used herein. It will be appreciated that a great variety of modifications have been made to DNA and RNA that serve many useful purposes known to those of skill in the art. The term “nucleic acid chains” as it is employed herein embraces such chemically, enzymatically or metabolically modified forms of polynucleotides, as well as the chemical forms of DNA and RNA characteristic of viruses and cells, including, for example, simple and complex cells. “Nucleic acid chains” also embraces short polynucleotides often referred to as oligonucleotide(s).

The term “polypeptide(s)” is art recognized and refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as “proteins”. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications may occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2^{nd} Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

The term “virus” is art-recognized and refers to a noncellular microbial entity that consists of a core of RNA or DNA enclosed in an outer coat of protein and, in some forms, a protective outer membrane, and that may live an reproduce only in susceptible host cells. Viruses may infect, for example, bacteria, plants, and animals, and more than 200 types have been identified as capable of causing diseases in humans, such as influenza, the common cold, measles, smallpox, and herpes.

The term “plasmid” is art-recognized and refers to a small, closed entity of double-stranded DNA forming an extra chromosomal self-replicating genetic element in many bacteria and some eukaryotes, often carrying genetic sequences that give the host cell a survival advantage such as resistance to antibiotics; plasmids are widely used in genetic engineering as a cloning vector.

The term “vector” is art-recognized and refers to any DNA molecule that may incorporate foreign DNA and transfer it from one organism to another.

The term “genetic engineering” is art-recognized and refers to altering the genome of a living cell for medical or industrial use.

The term “cell” is art-recognized and refers to the structural and functional unit of all living organisms. Some organisms, such as bacteria, are unicellular, consisting of a single cell. Other organisms, such as humans, are multicellular. The cells may be prokaryotic or eukaryotic, unicellular or multicellular, and may originate from any of the five kingdoms of cells: monera, protista, fungi, plantae, and animalia. At the cellular level, there are changes from kingdom to kingdom.

The term “eukaryotic” is art-recognized and refers to an organism whose cells have a distinct nucleus, multiple chromosomes, and a mitotic cycle; this classification thus includes animals, plants, and fungi, but not bacteria or algae.

The term “prokaryotic” is art-recognized and refers to an organism of the kingdom Procaryotae, including the bacteria and cyanobacteria; characterized by the lack of defined nucleus, and the possession of a single double-stranded DNA molecule and a very small range of organelles.

The term “tissue” is art-recognized and refers to a group of similarly specialized cells that perform a common function; tissues compose the organs and other structures of living organisms.

An “imaging agent” shall mean a composition capable of generating a detectable image upon binding with a target and shall include radionuclides (e.g., In-111, Tc-99m, I-123, I-125 F-18, Ga-67, Ga-680); for Positron Emission Tomography (PET) and Single Photon Emission Tomography (SPECT), unpair spin atoms and free radicals (e.g., Fe, lanthanides, and Gd); and contrast agents (e.g., chelated (DTPA) manganese) for Magnetic Resonance Imaging (MRI). Imaging agents are discussed in greater detail below.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which may be screened with any of the assays used in various embodiments herein. The term “small organic molecule” refers to a small molecule that is often identified as being an organic or medicinal compound, and does not include molecules that are exclusively nucleic acids, peptides or polypeptides.

The term “modulation” is art-recognized and refers to up regulation (i.e., activation or stimulation), down regulation (i.e., inhibition or suppression) of a response, or the two in combination or apart.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the embodiments that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, coordination complexes of various embodiments.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject supplement, composition, therapeutic or other material other than directly into the central nervous system, such that it enters the patient's system and, thus, is subject to metabolism and other like processes, for example, subcutaneous administration.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

The terms “transporting materials” and “transportable materials” as used herein refer to substances that are capable of being introduced into biological cells via the cell membranes and/or cell walls of the biological cells when the cell membranes and/or cell walls are disrupted with sonic energy.

The term “ex-vivo” as used herein refers to being outside of the body or living organism.

The term “viable” as used herein refers to being alive or capable of being alive.

The term “viable organism” as used herein refers to a multi-cellular living organism or a multi-cellular organism that is capable of being alive.

The term “mix” as used herein refers to a fluid composition of two or more substances, possibly including biological cells, materials to be transported into the biological cells, and a medium.

The term “transport characteristic of cell membranes” as used herein refers to those characteristics of cell membranes of biological cells which affect the transportation of materials into the biological cells through the cell membranes.

The term “substantially singular type” as used herein refers to a sample of biological cells or materials that are dominated by a particular kind of biological cell or a particular kind of material, allowing for the possibility that there may be some relatively small amount of other biological cells or other materials present in the sample.

The term “transportation qualities” as used herein refers to those characteristics of a mix of biological cells and materials that affect the transportation of the materials into the biological cells via the cell membranes and/or cell walls of the biological cells.

The term “transport efficiency” as used herein refers to how well materials are transported into biological cells via disruption of cell membranes and/or cell walls of the biological cells when exposed to sonic energy having particular parameters.

A method of transporting materials into biological cells ex-vivo using sonic energy is disclosed. The method includes extracting a sample of viable biological cells from a human subject and preparing a mix comprising the sample of viable biological cells and materials to be transported. The method further includes exposing the mix to sonic energy of a pre-determined target energy level to isothermally change a transport characteristic of cell membranes and/or cell walls of the biological cells such that at least a portion of the materials transport into at least a portion of the biological cells and such that at least a portion of the biological cells remain viable. The method also includes terminating the exposing after a pre-determined exposure time such that the transported materials become trapped within the biological cells. The method further includes re-introducing the viable biological cells into the human subject.

A first embodiment of a method of introducing material into biological cells using sonic energy is disclosed. The method includes preparing a mix comprising biological cells and materials to be transported into the biological cells. The method further includes selecting a sonic energy waveform corresponding to a first set of sonic energy parameters previously associated with the biological cells and the materials based on transportation characteristics of the materials into the biological cells. The method also includes exposing the mix to sonic energy, according to the selected waveform having the first set of sonic energy parameters, to change a transport characteristic of cell membranes and/or cell walls of the biological cells such that at least a portion of the materials are transported through the cell membranes and/or cell walls into at least a portion of the biological cells without substantially damaging the biological cells. The method further includes terminating the exposing to trap the transported materials within the biological cells.

A second embodiment of a method of introducing materials into biological cells using sonic energy is disclosed. The method includes preparing a sample, wherein the sample comprises biological cells and materials to be transported into the biological cells. The method further includes exposing the sample to sonic energy wherein the sonic energy is supplied at about a 1% duty cycle, at about 10 to 50 cycles per burst, at about 100 to about 300 mV, and for about 5 to about 10 seconds.

A method of correlating sonic energy exposure to transport efficiency in biological cells is disclosed. The method includes:
(a) exposing a solution to a level of sonic energy for a period of time, where the solution comprises biological cells and materials to be transported into the biological cells;
(b) examining the biological cells, after the exposing, for transport of the materials into the cells and for cell damage;
(c) determining and recording an amount of transported materials at the level of sonic energy for the period of time in response to the examining;
(d) increasing the level of sonic energy and/or the period of time and repeating steps (a) through (d) if no cell damage was observed in step (b); and
(e) determining the sonic energy and/or period of time for substantially optimized transport efficiency relating to the type of biological cells and type of materials to be transported into the type of biological cells from step (d).

A third embodiment of a method of introducing materials into biological cells using sonic energy is disclosed. The method includes preparing a sample in a treatment vessel, wherein the sample comprises biological cells and materials to be transported into the biological cells. The method further includes exposing the sample to a first sonic energy corresponding to a lower power mixing mode to sonically stir the biological cells and the materials of the sample. The method also includes exposing the sample to a second sonic energy corresponding to a higher power treatment mode to change a transport characteristic of cell membranes and/or cell walls of the biological cells such that at least a portion of the materials transport into the biological cells.

A method of introducing materials into viable organisms using sonic energy is disclosed. The method includes preparing a sample in a treatment vessel, wherein the sample comprises viable organisms, a viable organism medium, and transportable materials. The method further includes exposing the sample to a pre-defined sonic energy waveform such that at least a portion of the transportable materials are transported into cells of the viable organisms via cell membranes and/or cell walls of the cells when a transport characteristic of the cells is changed by the exposing sonic energy waveform.

These and other advantages and novel features of the present invention, as well as details of illustrated embodiments thereof, will be more fully understood from the following description and drawings.

### BRIEF DESCRIPTION OF THE DRAWINGS

FIG. 1 depicts a schematic method for delivering materials into biological systems using sonic energy;

FIG. 2 is a flowchart of an embodiment of a method of transporting materials into biological cells ex-vivo using sonic energy;

FIG. 3 is a flowchart of a first embodiment of a method of introducing materials into biological cells using sonic energy;

FIG. 4 is a flowchart of a second embodiment of a method of introducing materials into biological cells using sonic energy;

FIG. 5 is a flowchart of an embodiment of a method of correlating sonic energy exposure to transport efficiency in biological cells;

FIG. 6 is a table showing the correlation between particle size, barrier, and minimal energy required for high transfection efficiency with improved viability;

FIGS. 7A-7B is a table summarizing flowcytometry results of transfected 293T cells;

FIG. 8 is a table summarizing flowcytometry results of transfected C6/36 cells;

FIG. 9 is a table summarizing RT-PCR reactions of transfected adult Hydra;

FIGS. 10A-10C is a table summarizing treatments used in IFA analysis;

FIG. 11 is a table summarizing treatments used in E. Coli transformations;

FIG. 12 is a flowchart of a third embodiment of a method of introducing materials into biological cells using sonic energy; and

FIG. 13 is a flowchart of an embodiment of a method of introducing materials into viable organisms using sonic energy.

### DETAILED DESCRIPTION OF THE INVENTION

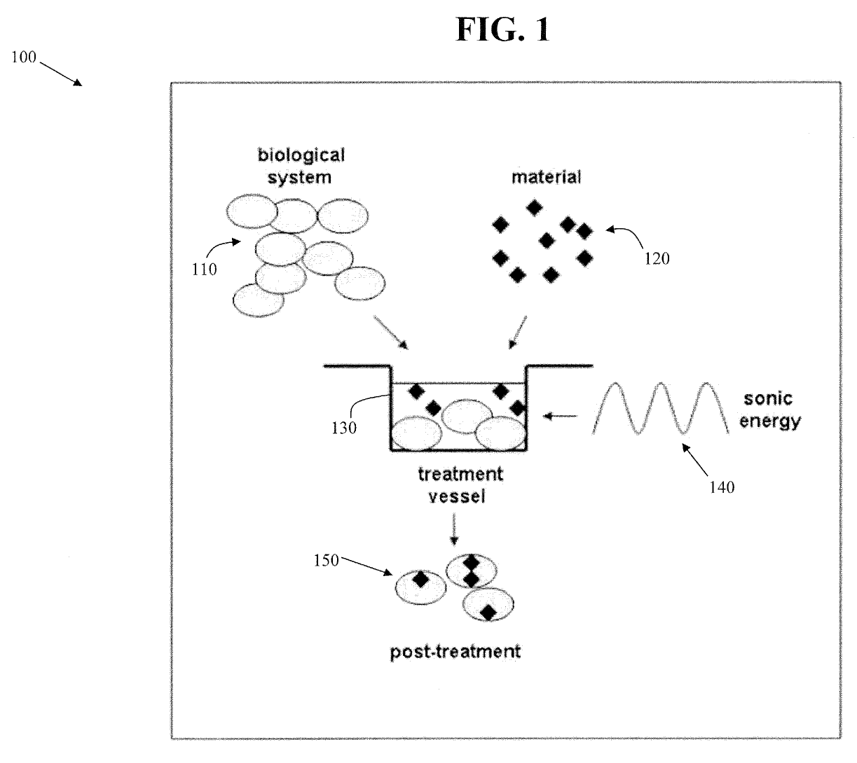

The concept of temporarily disrupting cell membranes and/or cell walls in biological cells is useful when trying to transport particles or materials into the biological cells. FIG. 1 depicts a schematic method 100 for delivering materials into biological systems using sonic energy. A sample of biological cells may be prepared, for example, in-vitro in a treatment vessel, or may be prepared, for example, ex-vivo by extracting the cells from a patient's body. A sample of biological cells 110 (e.g., human skin cells) are identified and selected along with materials 120 (e.g., a protein) and are placed in a treatment vessel 130 possibly along with a fluid (e.g., water) to form a mix or solution of the biological cells 110 and the material 120. Sonic energy 140, having certain characteristics (e.g., a duty cycle, a number of cycles per burst, a peak amplitude, and an exposing time duration) is used to treat the mix of cells 110 and material 120 via exposure.

When the mix of cells 110 and materials 120 are exposed to the sonic energy 140, the membranes and/or walls of the cells 110 are disrupted (e.g., pores open in the membranes/walls) such that the materials 120 may enter the cells 110 through the membranes/walls. When the exposure to the sonic energy 140 is stopped, the disruption stops (e.g., the pores close), trapping the materials 120 inside the cells 110, forming populated cells 150. The characteristics of the sonic energy 140 are such that the resultant disruption of the membranes/walls is sufficient for the material 120 to transport into the cells 110.

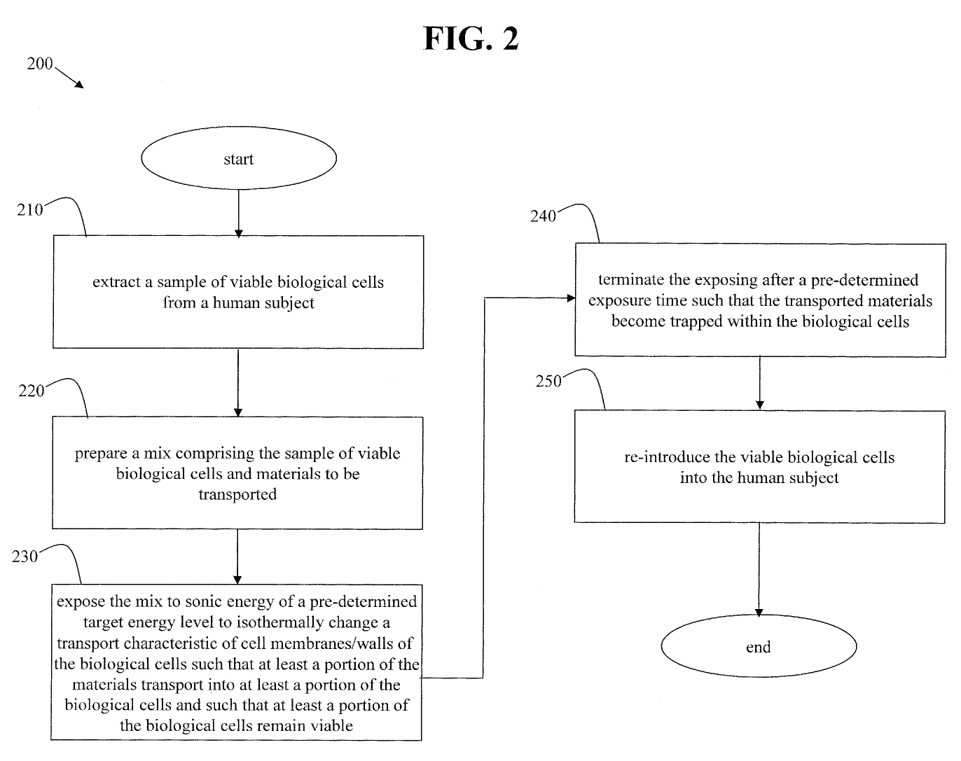

FIG. 2 is a flowchart of an embodiment of a method 200 of transporting materials into biological cells ex-vivo using sonic energy. In step 210, a sample of viable biological cells are extracted from a human subject. In step 220, a mix is prepared comprising the sample of viable biological cells and materials to be transported. In step 230, the mix is exposed to sonic energy of a pre-determined target energy level to isothermally change a transport characteristic of cell membranes and/or cell walls of the biological cells such that at least a portion of the materials transport into at least a portion of the biological cells and such that at least a portion of the biological cells remain viable. In step 240, the exposing is terminated after a pre-determined exposure time such that the transported materials become trapped within the biological cells. In step 250, the viable biological cells are re-introduced into the human subject.

The biological cells may be of a substantially singular type or may be a combination of various types of cells. Similarly, the materials may be of a substantially singular type or may be a combination of various types of materials. The materials may include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

The parameters of the exposing sonic energy may include an ultrasonic frequency, a duty cycle, a number of cycles per burst, a peak amplitude, and an exposing time duration. For example, the parameters of the exposing sonic energy may comprise about a 1% duty cycle, at about 10 to 50 cycles per burst, at about 100 to 300 millivolts, for about 5 to 10 seconds.

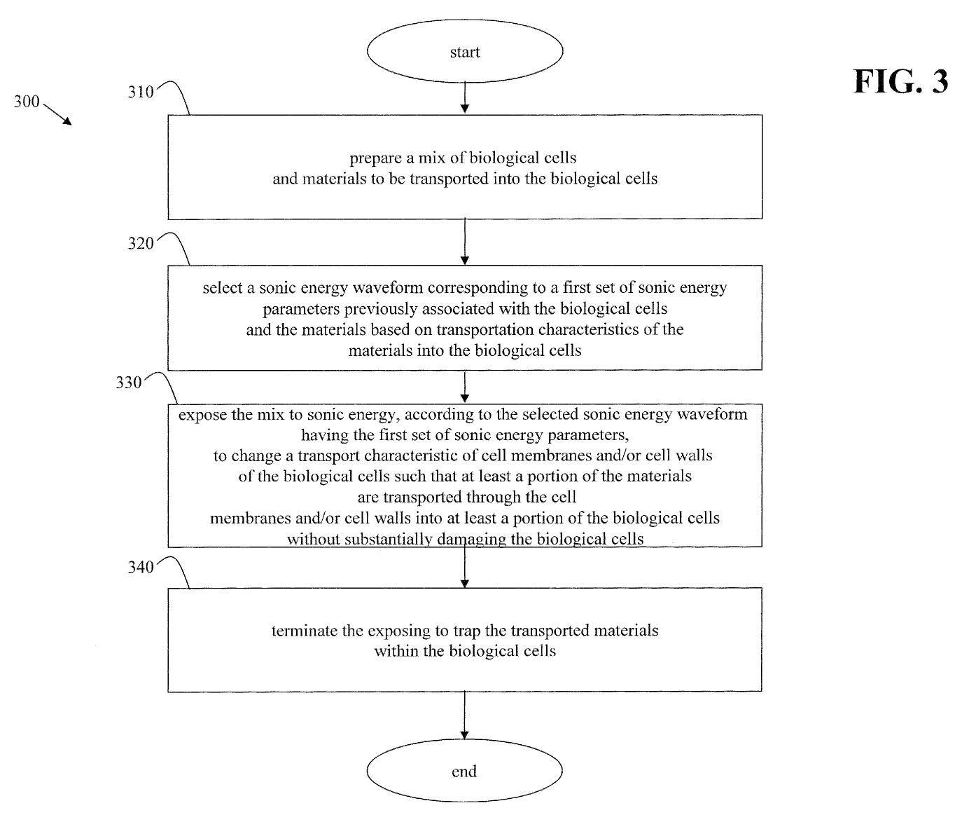

FIG. 3 is a flowchart of a first embodiment of a method 300 of introducing materials into biological cells using sonic energy. In step 310, a mix of biological cells and materials to be transported into the biological cells is prepared. The mix may be prepared in-vitro (e.g., in a treatment vessel 130). The biological cells may be a singular type of biological cells or may be some combination of different types of biological cells. Similarly, the materials may be biological or non-biological and may be of a singular type or some combination. Again, for example, the materials may comprise at least one of chemicals, inorganic molecules, organic molecules, monomers, nucleic acids, amino acids, polymers, nucleic acid chains, proteins, viruses, plasmids, and vectors. The materials may tend to be of a certain size (e.g., a certain diameter) and/or shape (e.g., round or spherical). Furthermore, the materials may comprise DNA, RNA, other nucleic acid constructs, nucleic acid monomers, monomers including saccharides, polymers including polysaccharides, amino acid chains, enzymes, cofactors, and/or visualization reagents such as fluorescent probes.

In step 320, a sonic energy waveform is selected which corresponds to a first set of sonic energy parameters previously associated with the biological cells and the materials based on transportation characteristics of the materials into the biological cells. In step 330, the mix is exposed to sonic energy, according to the selected sonic energy waveform having the first set of sonic energy parameters, to change a transport characteristic of cell membranes and/or cell walls of the biological cells such that at least a portion of the materials are transported through the cell membranes and/or cell walls into at least a portion of the biological cells without substantially damaging the biological cells. In step 340, the exposing is terminated to trap the transported material within the biological cells.

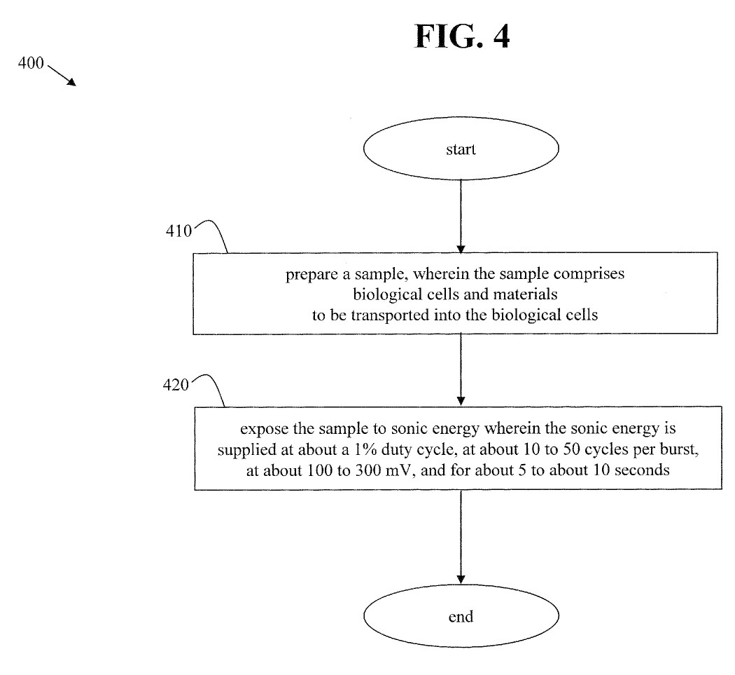

Again, the sonic energy parameters may include an ultrasonic frequency, a duty cycle, a number of cycles per burst, a peak amplitude, and an exposing time duration. For Example, FIG. 4 is a flowchart of a second embodiment of a method 400 of introducing materials into biological cells using sonic energy. In step 410, a sample comprising biological cells and materials to be transported into the biological cells is prepared. In step 420, the sample is exposed to sonic energy wherein the sonic energy is supplied at about a 1% duty cycle, at about 10 to 50 cycles per burst, at about 100 to 300 millivolts (mV), and for about 5 to 10 seconds.

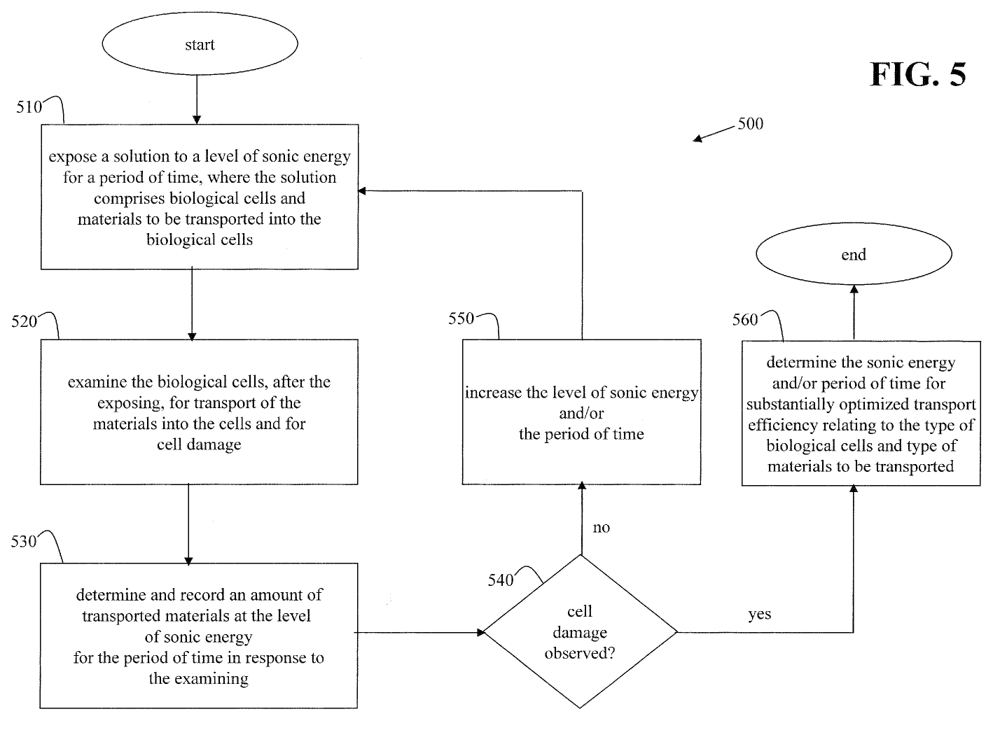

In order to know what sonic energy allows for disrupting of the cell membranes and/or cell walls of what types of cells, and to what extent, experiments are first performed. For example, FIG. 5 is a flowchart of an embodiment of a method 500 of correlating sonic energy exposure to transport efficiency in biological cells. In step 510, a solution is exposed to a level of sonic energy for a period of time, where the solution comprises biological cells and materials to be transported into the biological cells. In step 520, the biological cells are examined, after the exposing, for transport of the materials into the cells and for cell damage. In step 530, an amount of transported materials are determined and recorded at the level of sonic energy for the period of time in response to the examining. In step 540, it is acknowledged if any cell damage was observed during the examination step. If no or minimal cell damage was observed then, in step 550, the level of sonic energy and/or the period of time are increased and the method 500 is repeated. If some unacceptable or barely acceptable level of cell damage is observed then, in step 560, the iterative part of the process is complete and determining the sonic energy and/or period of time for substantially optimized transport efficiency relating to the type of biological cells and type of materials to be transported into the type of biological cells from step 540 is performed. Ideally, the exposing to sonic energy is accomplished in an isothermal manner before cell damage may occur.

For example, the method 500 may start out at a relatively low level of sonic energy which is stepped up some amount for each iteration of the method 500 until cell damage, or a particular percentage of cells damaged, is observed. As a result, an experimenter may determine the various levels of sonic energy which disrupt the cell membranes and/or cell walls such that efficient transport may occur including, for example, an optimum level and/or period of time. Once such information is known, an experimenter may know which materials may be transported into the biological cells and how efficiently. The materials may be actual materials that are desired to be transported into the biological cells, or the materials may be “dummy” materials (e.g., synthetic substances) having certain shapes and sizes which mimic actual materials that are desired to be used.

The methods described herein are based in part on the use of controlled sonic energy to isothermally disrupt cellular membranes and/or cell walls allowing entry of materials into cells and tissues.

Cells, tissues, and whole organisms may be treated with sonic energy while in their normal growth or culture medium without having to put them in a medium that may not be biologically compatible. As sonic energy may be delivered isothermally, cells, tissues, and whole organisms may be maintained at their normal incubation temperatures during treatment, facilitating recovery after membrane and cell wall disruption. During treatment, cells, tissues, or organisms are mixed with the desired material to be delivered in normal growth, culture, or desired medium in treatment vessels such as borosilicate tubes or polystyrene cell culture plates in various formats including 6-well, 12-well, 24-well, or 96-well plates or other vessels. To confirm material delivery into target cells, tissues, or organisms, appropriate methods are used including but not limited to microscopy, flow cytometry, biological, chemical, and molecular assays. Embodiments described herein may be applied to fields including but not limited to medicine, drug discovery, pharmaceutical, biotechnology, gene therapy, stem cell research, cancer research and treatment, developmental biology, cell biology, organismal biology, physiology, chemistry, biochemistry, microbiology, molecular biology, botany, bacteriology, virology, developing transgenic organisms, veterinary science, agriculture, and basic and applied scientific research, for example.

Certain embodiments described herein provide a controlled manner in which sonic energy may be administered to cells. By “controlled manner” it is meant that various experimental parameters may be adjusted and set as needed for a particular cell type and/or material to be inserted. These parameters include, but are not limited to, temperature and energy (i.e. cycles/burst, amplitude, duration time, and the like). Additionally, the methods allows for a very focused application of sonic energy as opposed to the broad application of a sonicator, for example. The sum effect of these abilities is greater reproducibility which not only assists in transfected cell production, but in scientific study in this area.

The introduction of materials into biological samples with high transfection efficiency and improved viability by minimizing the amount of energy required to introduce material to the biological samples is described herein. This discovery embodies the fact that the isothermal method maintains the biological samples at their physiologically relevant temperatures so that the samples do not overheat or degrade. Membrane pore openings are transient in nature and allow the delivery of nanoparticles of various sizes into biological samples with minimal toxicity. Surprisingly, it has been found that once sufficient energy is applied to open a pore in a cell membrane, very little additional energy is required to further increase the size of the pore. One would logically expect that a larger particle would require more energy to form an adequate pore size in the cell membrane than would be required for a smaller particle size. However, it has been determined that using larger amounts of energy than necessary often leads to cell deterioration rather than high transfection efficiencies. The size of the particle is of minimal consequence as long as it is small enough not to damage the target cell. It has now been discovered that the composition and physical properties of the barrier, for example, whether it is a cell plasma membrane or a bacterial cell wall, determine the amount of energy required for formation of a pore in that barrier. Introduction of particles into biological materials with additional barriers may require additional energy for efficient delivery.

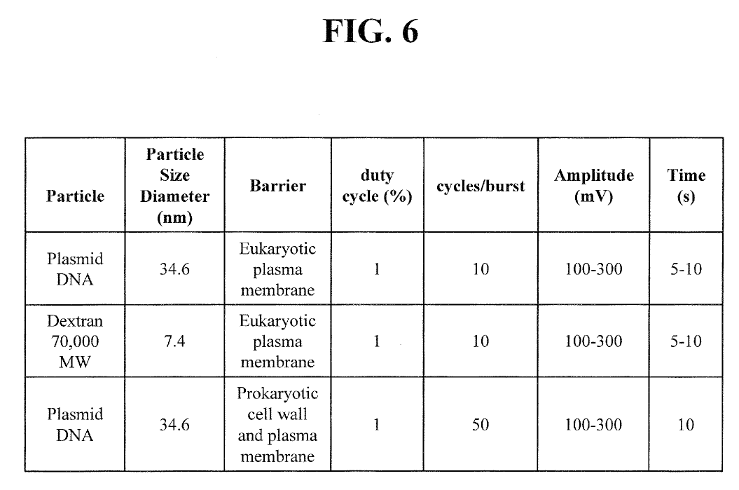

Such discoveries may be seen in the entries of the table shown in FIG. 6. FIG. 6 is a table showing the correlation between particle size, barrier, and minimal energy required for high transfection efficiency with improved viability. First, note how unexpectedly the same amount of energy was used to introduce plasmid DNA into eukaryotic cells as that for dextran even though the particle size of plasmid DNA is 4.5 times greater. Second, note that greater energy was necessary to introduce plasmid DNA into prokaryotic cells than eukaryotic cells (50 cycles/burst v. 10 cycles/burst) because of the additional plasma membrane barrier.

Because all cells have a protective membrane comprising lipids and proteins, the methods described herein are equally effective on all cell types. A component of every biological cell, the selectively permeable cell membrane (or plasma membrane or plasmalemma) is a thin and structured bilayer of phospholipid and protein molecules that envelopes the cell. It separates a cell's interior from its surroundings and controls what moves in and out. Cell surface membranes often contain receptor proteins and cell adhesion proteins. There are also other proteins with a variety of functions. These membrane proteins are important for the regulation of cell behavior and the organization of cells in tissues.

In animal cells, the cell membrane establishes this separation alone, whereas in yeast, bacteria and plants an additional cell wall forms the outermost boundary, providing primarily mechanical support. The plasma membrane is only about 10 nm thick and may be discerned only faintly with a transmission electron microscope. One of the roles of the membrane is to maintain the cell potential.

The basic composition and structure of the plasma membrane is the same as that of the membranes that surround organelles and other subcellular compartments. The foundation is a phospholipid bilayer, and the membrane as a whole is often described as a fluid mosaic—a two-dimensional fluid of freely diffusing lipids, dotted or embedded with proteins, which may function as channels or transporters across the membrane, or as receptors.

Some of these proteins simply adhere to the membrane (extrinsic or peripheral proteins), whereas others might be said to reside within it or to span it (intrinsic proteins—more at integral membrane protein). Glycoproteins have carbohydrates attached to their extracellular domains. Cells may vary the variety and the relative amounts of different lipids to maintain the fluidity of their membranes despite changes in temperature. Cholesterol molecules (in case of eukaryotes) or hopanoids (in case of prokaryotes) in the bilayer assist in regulating fluidity.

Phospholipid molecules in the cell membrane are “fluid,” in the sense of free to diffuse and exhibit rapid lateral diffusion. Lipid rafts and caveolae are examples of cholesterol-enriched microdomains in the cell membrane. Many proteins are not free to diffuse. The cytoskeleton undergirds the cell membrane and provides anchoring points for integral membrane proteins. Anchoring restricts them to a particular cell face or surface—for example, the “apical” surface of epithelial cells that line the vertebrate gut—and limits how far they may diffuse within the bilayer. Rather than presenting always a formless and fluid contour, the plasma membrane surface of cells may show structure. With epithelial cells in the gut, for example, the apical surfaces of many such cells are dense with involutions, all similar in size. The finger-like projections, called microvilli, increase cell surface area and facilitate the absorption of molecules from the outside. Synapses are another example of highly-structured membrane.

New material is incorporated into the membrane by a variety of mechanisms: (i) fusion of intracellular vesicles with the membrane not only excretes the contents of the vesicle, but also incorporates the vesicle membrane's components into the cell membrane; (ii) if a membrane is continuous with a tubular structure made of membrane material, then material from the tube may be drawn into the membrane continuously; and (iii) although the concentration of membrane components in the aqueous phase is low (stable membrane components have low solubility in water), exchange of molecules with this small reservoir is possible. In all cases, the mechanical tension in the membrane has an effect on the rate of exchange. In some cells, usually having a smooth shape, the membrane tension and area are interrelated by elastic and dynamical mechanical properties, and the time-dependent interrelation is sometimes called homeostasis, area regulation or tension regulation. These mechanisms, however, are limited by the size and lipophilicity of the material as discussed below.

As a lipid bilayer, the cell membrane is semi-permeable. This means that only some molecules may pass unhindered in or out of the cell. These molecules are either small or lipophilic. Other molecules may pass in or out of the cell, if there are specific transport molecules. Depending on the molecule, transport occurs by different mechanisms, which may be separated into those that do not consume energy in the form of ATP (passive transport) and those that do (active transport). By using sonic energy as disclosed herein, transfected material is not limited by size, lipophilicity, or the need of a specific transport. Therefore, the present method works equally well with a wide variety of cells.

The cells may be prokaryotic or eukaryotic, unicellular or multicellular, and may originate from any of the five kingdoms of cells: monera, protista, fungi, plantae, and animalia. At the cellular level, there are changes from kingdom to kingdom.

Moneran cells are prokaryotic and may be unicellular or multicellular. They possess a cell wall and the genetic material is found in the cytoplasm which lacks membrane-bound organelles. Some have a flagellum and they engulf food from outside sources. Examples of moneran cells include bacteria and blue-green algae.

Protist cells are eukaryotic and may be unicellular or multicellular. Many protist cells are animal or plant-like (some contain chlorophyll). They possess a flagellum, which may be used for locomotion, and they produce their own food, but also capture food from outside sources. Examples include paramecium, euglena, diatoms, golden algae, dinoflagellates, green algae, brown algae, and red algae.

Fungi cells are eukaryotic and may be unicellular or multicellular. Fungi cell walls are made of chitin and are not sealed which allows cytoplasm to be transferred from cell to cell. There is more than one nuclei per cell, and they obtain food from outside sources. Examples include yeast, mushrooms, penicillin molds, lichens, and slime molds.

Plant cells are eukaryotic and multicellular. The cell walls are made of cellulose and plant cells comprise numerous plastids. They have characteristically large vacuoles and they produce their own food. Examples include mosses, fir trees, and flowering plants.

Animal cells are eukaryotic and multicellular. There are two main types of animal cells: vertebrates and invertebrates. They take in organic matter for food and examples include sponges, jellyfish, worms, spiders, snails, crabs, insects, fish, birds, and mammals, such as humans.

Materials that may be introduced into cells by the present methods include biological or non-biological materials. These materials include, but are not limited to DNA, RNA, other nucleic acid constructs, nucleic acid monomers, plasmids, vectors, viruses, monomers including saccharides, polymers including polysaccharides, amino acids, amino acid chains, enzymes, polymers, organic molecules, inorganic molecules, proteins, cofactors, and/or visualization reagents such as fluorescent probes.

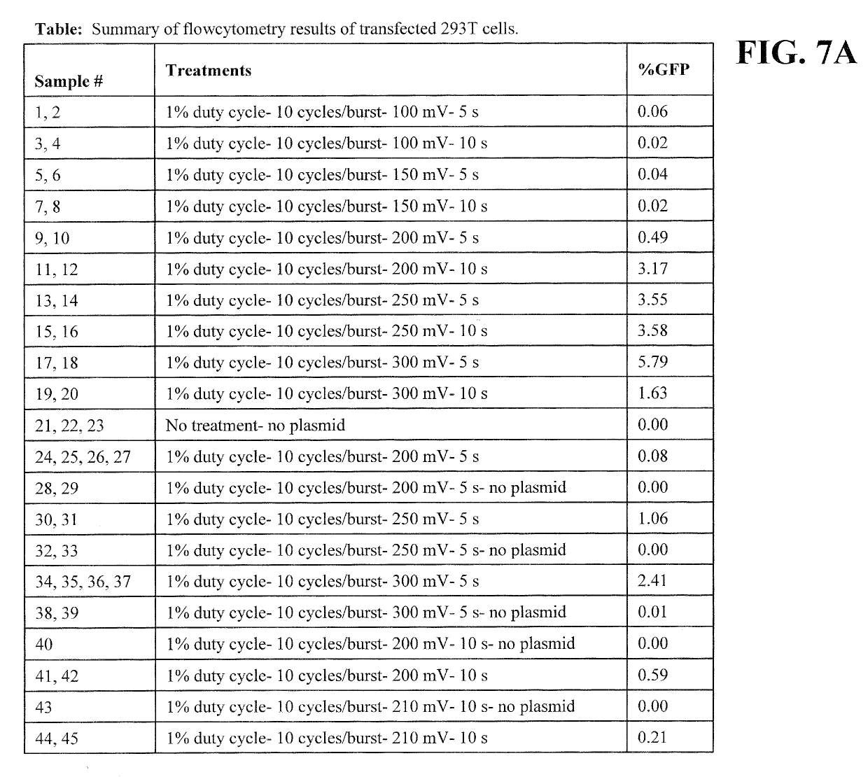

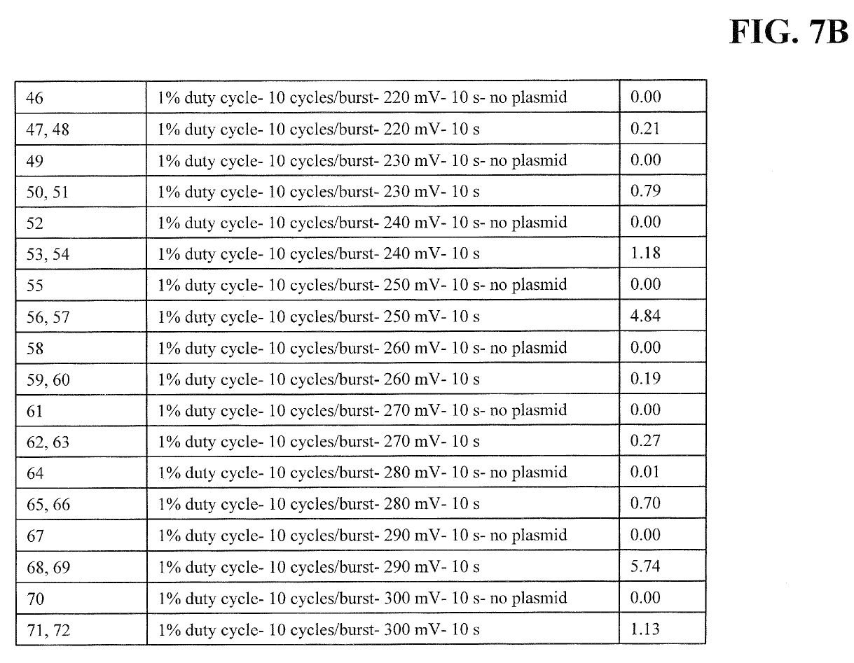

An example is the transfection of human embryonic kidney cells with plasmid DNA using focused sonic energy. For each transfection, human embryonic kidney cells 293T (American Type Culture Collection, Manassas, Va.) in plastic 60 mm or 100 mm plates cultured at 37° C./5% CO2 in cell culture medium (Dulbecco's modified Eagle's medium (Invitrogen, Carlsbad, Calif.) supplemented with 10% v/v fetal bovine serum (Invitrogen), 292 μg/ml g lutamax (Invitrogen), 100 units/ml penicillin (Invitrogen), and 100 μg/ml streptomycin (Invitrogen)) were resuspended in 500 μl of cell culture medium and transferred to sterile 13×65 mm, 5 ml borosilicate tubes (Chromacol Ltd., Herts, United Kingdom). All transfections were performed in duplicate. To each tube, approximately 4 μg of plasmid DNA, pEGFP-C1 (Clontech, Mountain View, Calif.) which contains a cytomegalovirus immediate early promoter driving expression of enhanced green fluorescent protein were added and placed in the Covaris E200 instrument (Covaris Inc., Woburn, Mass.) 4×6 array sample holder plate. All transfections were performed at 37° C. to maintain normal cell incubation temperature. Cells were subjected to various treatments as described in the table of FIGS. 7A-7B. Twenty-four and 48 h post transfection, cells were analyzed for gene expression using fluorescence microscopy using an Olympus CK 41 inverted microscope with a fluorescence module (Olympus, Melville, N.Y.) and/or flowcytometry using a Cytomics FC 500 (Beckman-Coulter, Fullerton, Calif.). In preparation for flowcytometry, 293T were resuspended 400 μl of cell culture medium to which 200 μl of sodium heparin (stock 10,000 U/ml) (Sigma-Aldrich, St. Louis, Mo.) to prevent cell clumping, and incubated on ice until analysis. Cytotoxicity was observed with increased mV. Increase in cytotoxicity correlated to increased levels of EGFP (Enhanced Green Fluorescent Protein) expression both by fluorescence microscopy and flowcytometry. At lower mV treatments, intact confluent monolayers were observed with few of the cells expressing EGFP. At the highest mV treatments, small to large cell patches were present in the plates with a large percentage of the cells in the patches expressing EGFP.

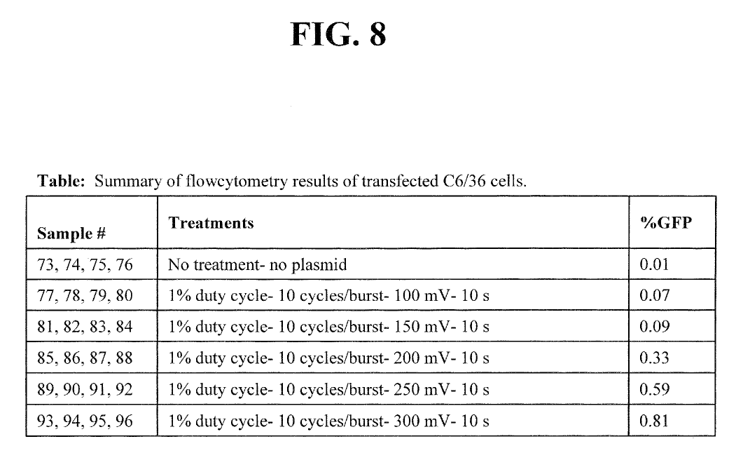

Another example is that of mosquito cells transfected with plasmid DNA using focused sonic energy. Immortalized Aedes albopictus (Asian tiger mosquito) cells C6/36 (American Type Culture Collection) in sterile 13×65 mm borosilicate tubes containing 500 μl of mosquito cell culture medium (Leibovitz's L15 medium (Invitrogen) supplemented with 10% v/v fetal bovine serum, 2 mM L-glutamine (Invitrogen), 1% v/v tryptose phosphate broth (29.2 mg/ml in 0.85% NaCl) (Sigma-Aldrich, St. Louis, Mo.), 100 units/ml penicillin, and 100 μg/ml streptomycin (Invitrogen)) were incubated with 20 μg of plasmid DNA, pBSII.ITR1.1K-EGFP/SV40DmAct5c containing the Drosophila melanogaster actin promoter driving expression of EGFP. All transfections were performed at 27° C. to maintain normal cell incubation temperature. Cells were subjected to various treatments in quadruplicate using the Covaris E200 instrument in 4×6 array sample holder plate as described in the table of FIG. 8. Twenty-four and 48 h post transfection, cells were analyzed for gene expression using fluorescence microscopy and/or flowcytometry. In preparation for flowcytometry, C6/36 cells were re-suspended in 400 μl of mosquito cell culture medium and 200 μl of sodium heparin (stock 10,000 units/ml) (Sigma-Aldrich) to prevent cell clumping, and incubated on ice until analysis. Cytotoxicity was observed with increased mV. Increase in cytotoxicity correlated to increased levels of EGFP expression both by fluorescence microscopy and flowcytometry.

A further example is that of the delivery of dextran-FITC (fluorescein isothiocyanate) into adult Hydra using focused sonic energy. To determine whether sonic energy using the Covaris E200 instrument could be used to deliver macromolecules to Hydra tissues, adult Hydra were placed in 13×65 mm, 5 ml borosilicate tubes in 500 μl of Hydra medium (1.0 mM CaCl2, 1.5 mM NaHCO3, 0.1 nM MgCl2, 0.08 mM MgSO4, 0.03 mM KNO3) containing 1 mM dextran-FITC 70,000 MW (Sigma-Aldrich). Samples were treated using the Covaris E200 at 18° C., the normal Hydra incubation temperature, under [1% duty cycle, 250 mV 10 cycles/burst 5 s], [1% duty cycle, 250 mV, 10 cycles/burst 10 s], [1% duty cycle, 300 mV 10 cycles/burst 5 s], [1% duty cycle 300 mV 10 cycles/burst 10 s] treatment conditions. Immediately after treatment, Hydra were rinsed thoroughly with Hydra medium to remove excess (uninternalized) dextran-FITC. Hydra were visualized using fluorescence microscopy. Intensity of FITC staining correlated with severity of treatment. Viability of Hydra was 100% for all treatments.

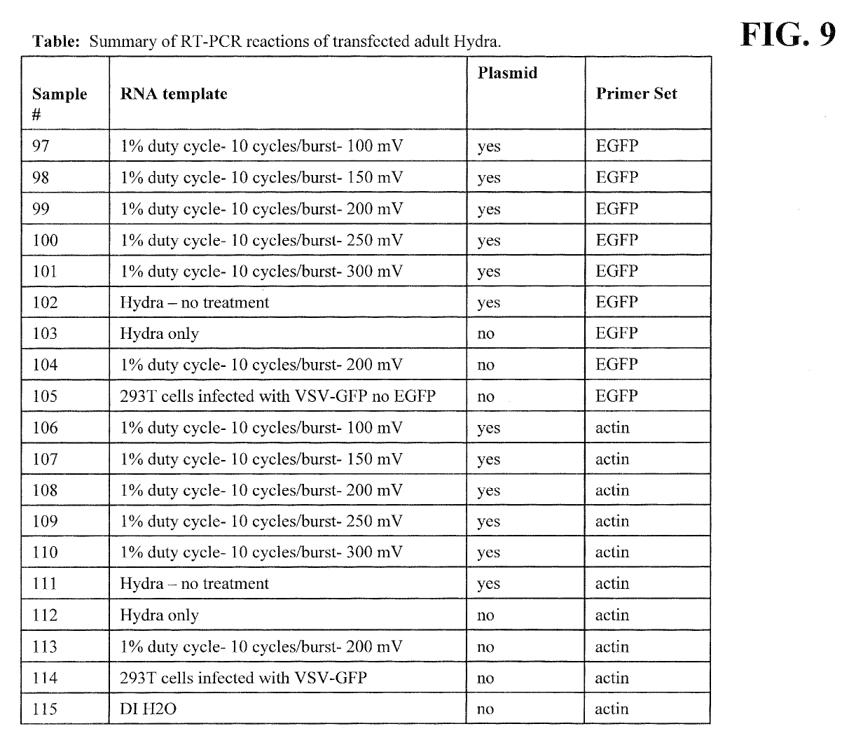

Another example is that of reverse transcription real time PCR on Hydra transfected with plasmid DNA using focused sonic energy (see the table of FIG. 9). Glass borosilicate tubes (13×65 mm, 5 ml; Chromacol Ltd., Herts, UK) containing 50 adult Hydra in Hydra medium were incubated with 15 μg of plasmid DNA containing a Hydra actin promoter driving EGFP (kindly provided by Hans Bode, University of California, Irvine) (“GFP expression in Hydra: lessons from the particle gun” Dev Genes Evol (2002) 212:302-305) and then subjected to various treatments using focused sonic energy with the Covaris E200 instrument. As a positive control, human embryonic kidney cells 293T were infected with 5 μl of a recombinant vesicular stomatitis virus encoding EGFP (VSV-GFP) and harvested 48 hours post-infection. Forty-eight hr. post treatment, total RNA was isolated using a High Pure RNA isolation kit (Roche Diagnostics, Chicago, Ill.), and reverse transcription reactions were prepared for real time PCR analysis using a LightCycler RNA Amplification kit SYBR green I (Roche Diagnostics) per manufacturer's instructions. Two amplification reactions were run with each template; to amplify EGFP (EGFPup primer: 5′-CCGGGTGGTGCCCATCCTGGTCG-3′, EGFPdown primer: 5′-CGGGCAGCAGCACGGGGCCGTCGCCG-3′) and endogenous actin (Actinup primer: 5′-TTCACCACTACAGCTGAGCGT-3′, Actindown primer: 5′-TGTTTGGAGATCCACATCTGTTGG-3′) fragments using 0.005 pmol of each primer. Reactions were performed in a Roche LightCycler under the following conditions: reverse transcription 55° C. for 30 min; denaturation 95° C. for 30 s; amplification for 45 cycles 95° C., 50° C. for 10 s, 72° C. for 20 s; melting curves 95° C., 65° C. for 10 s, 95° C.; cooling 40° C. for 30 s. Preliminary results suggest that EGFP mRNA transcripts could be detected using RT-PCR as indicated by the observed melting peaks.

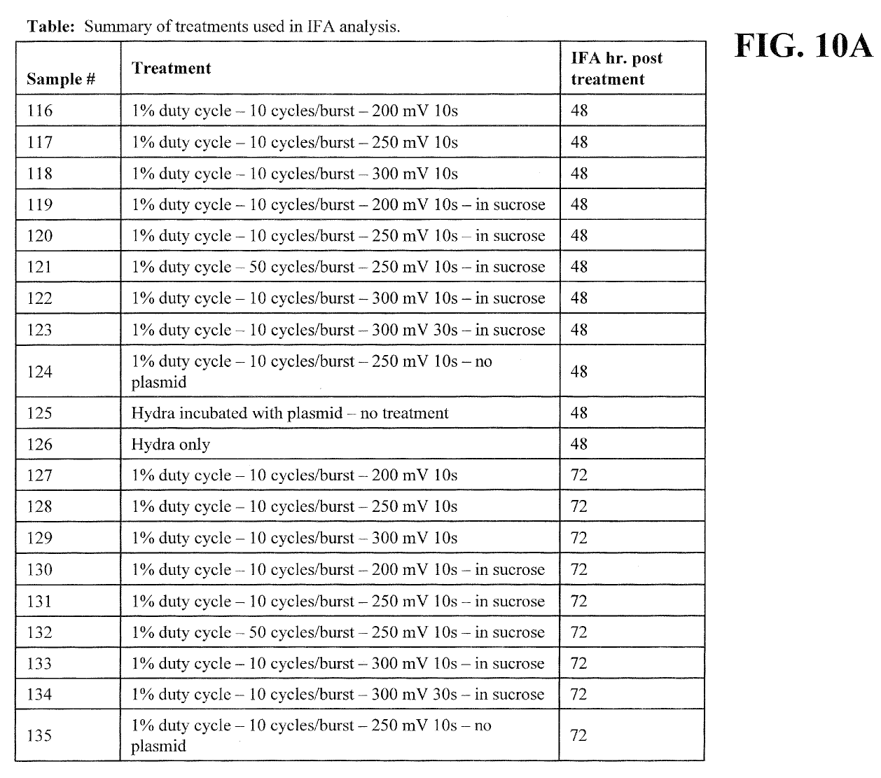

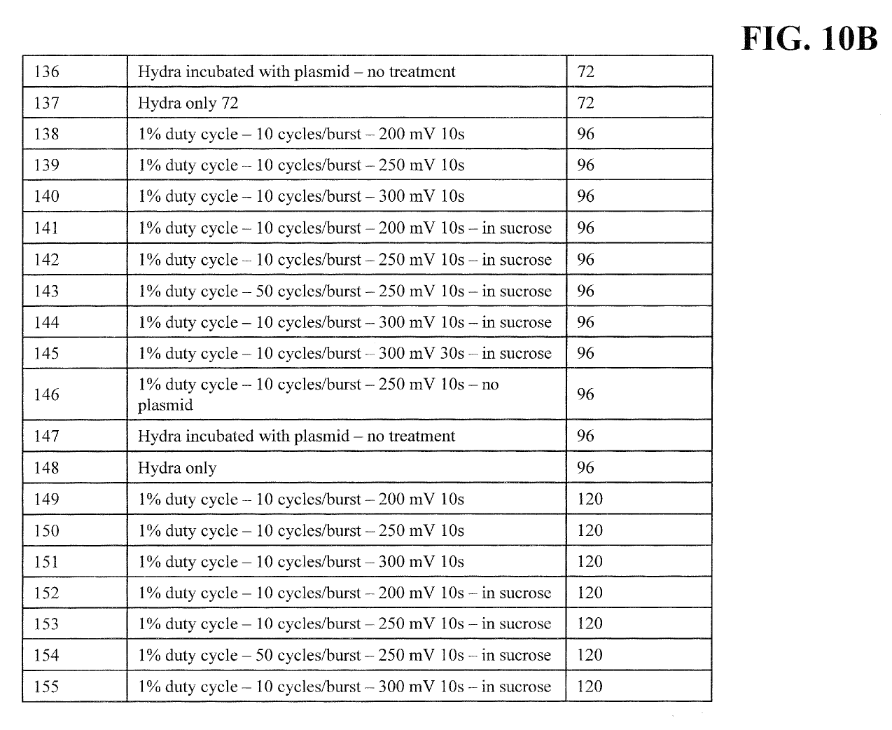

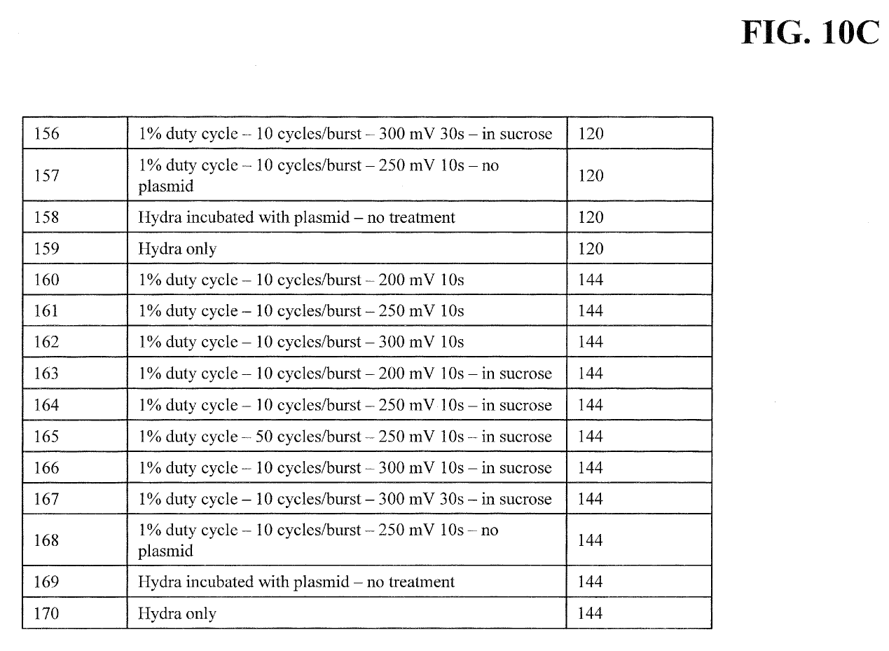

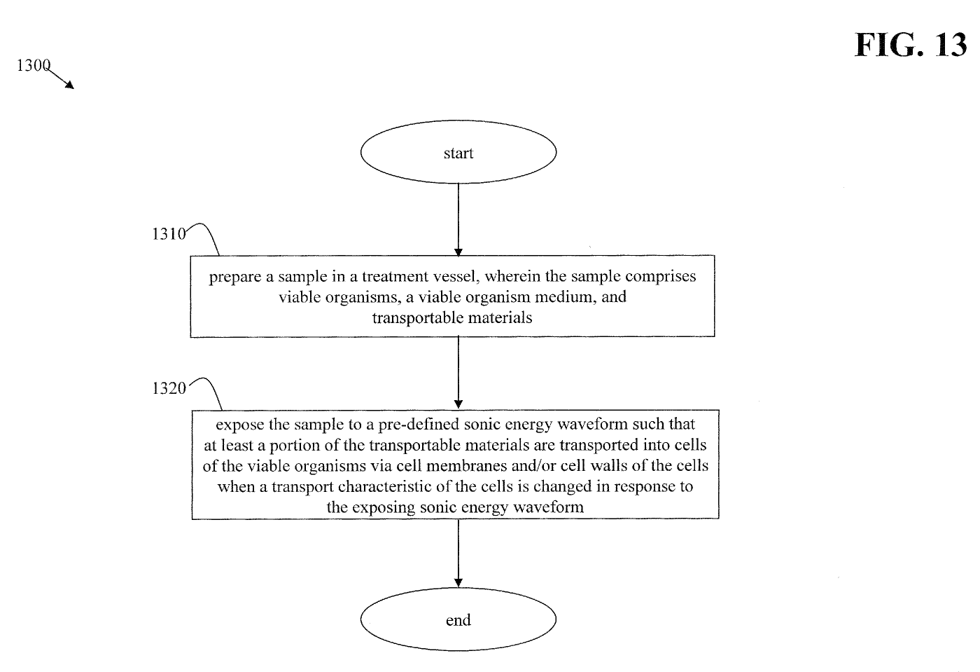

A further example is the immunofluorescence analysis of Hydra transfected with plasmid DNA using focused sonic energy (see the table of FIGS. 10A-10C). Glass borosilicate tubes (13×65 mm, 5 ml; Chromacol Ltd.) containing 6 adult hydra in 250 μl Hydra medium were incubated with 20 μg of plasmid DNA containing a Hydra actin promoter driving EGFP and then subjected to various treatments as described in the table of FIGS. 13A-13C on the Covaris E200 instrument. Prior to treatment, some samples of Hydra were incubated in a 50 mM sucrose solution in Hydra medium for 18 hours, as indicated below. Hydra were incubated in the dark at 18° C. following treatment. 48, 72, 96, 120, and 144 hr. post treatment the Hydra were assayed for EGFP expression using an immunofluorescent assay. Hydra were relaxed in a 2% w/v urethane (Fisher Scientific, Atlanta, Ga.) solution for 2 minutes. The Hydra were then fixed in Lavdowsky's fixative (50 parts 95% ethanol, 10 parts 37% formaldehyde, 4 parts glacial acetic acid, 36 parts deionized water) for 15 minutes. The fixed Hydra were incubated overnight at 4° C. with shaking in a 1:100 dilution of A.v. peptide antibody (rabbit anti-GFP protein) (Clontech, Mountain View, Calif.). Then Hydra were incubated with a 1:1000 dilution of Alexa Flour 488 goat anti-rabbit IgG secondary antibody (Molecular Probes, Eugene, Oreg.) for 3-4 hours at room temperature and then analyzed for EFGP protein amplification using fluorescence microscopy using an Olympus CK 41 inverted microscope with a fluorescence module. In sample numbers 120, 121, 128, 129, 131, 133, 139, 141, 151, and 153 at least one hydra of 6 expressed amplification of EGFP. All controls were negative for expression. Expression of EGFP appears transient and peaks between 72 and 120 hours after treatment. Morbidity of hydra increased as the treatment time increased from 10 to 30 seconds or as the cycles/burst increased from 10 to 50. At 1% duty cycle-50 cycles/burst-250 mV 10 s. and 1% duty cycle-10 cycles/burst-300 mV 30 s treatments the morbidity of the hydra was 100% by 144 hours after treatment. Expression of EGFP increased in the surviving hydra as morbidity increased.

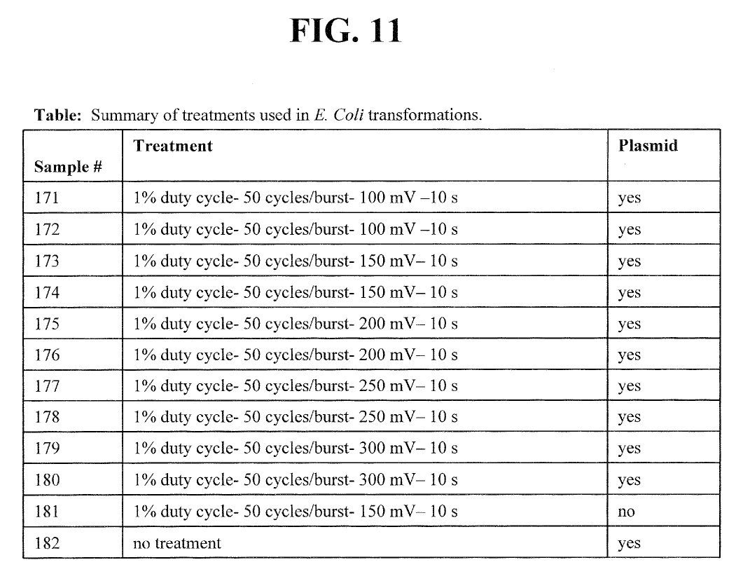

A further example is the transformation of E. Coli with plasmid DNA using focused sonic energy (see the table of FIG. 11). A 250 ml culture of E. Coli (STBL2) (Invitrogen) incubated overnight in LB medium in an incubator/shaker at 37° C., 175 r.p.m. was clarified by centrifugation at 3,800 r.p.m., at 4° C. for 10 min. Each pellet (the equivalent of 50 ml of starter culture) was re-suspended in 6 ml of S.O.C. medium (Invitrogen). Ten μg of plasmid DNA pNLEGFP/CMV (kindly provided by Jakob Reiser, Louisiana State University Health Sciences Center, New Orleans) were added to 500 μl aliquots of STBL2 cells in S.O.C. in sterile 13×65 mm, 5 ml borosilicate tubes (Chromacol Ltd.). Transformations were performed at 37° C. using the Covaris E200 under conditions indicated in Table 6. Following treatment, 50 μl of each transformation were plated on LB agar ampicillin plates and incubated overnight at 37° C. One colony was isolated from each plate and used to inoculate new LB agar ampicillin plates and incubated overnight at 37° C. Colonies were isolated and plasmid DNA was extracted and purified using a Qiagen Hispeed mini preparation plasmid DNA extraction kit (Qiagen, Valencia, Calif.). Restriction endonuclease analysis was performed to verify identity of isolated plasmid DNA. The band sizes of plasmid DNA from isolated colonies are consistent with the size of linearized control plasmid DNA. This suggests that sound energy using the Covaris E200 instrument may transform E. Coli with plasmid DNA.

In certain embodiments, the sonic energy source, for example, an ultrasound transducer or other transducer, produces acoustic waves in the “ultrasonic” frequency range. Ultrasonic waves start at frequencies above those that are audible, typically about 20,000 Hz or 20 kHz, and continue into the region of megahertz (MHz) waves. The speed of sound in water is about 1000 meters per second, and hence the wavelength of a 1000 Hz wave in water is about a meter, typically too long for specific focusing on individual areas less than one centimeter in diameter, although usable in non-focused field situations. At 20 kHz the wavelength is about 5 cm, which is effective in relatively small treatment vessels. Depending on the sample and vessel volume, desired frequencies may be higher, for example, about 100 kHz, about 1 MHz, or about 10 MHz, with wavelengths, respectively, of approximately 1.0, 0.1, and 0.01 cm. In contrast, for conventional sonication, including sonic welding, frequencies are typically approximately in the tens of kHz, and for imaging, frequencies are more typically about 1 MHz and up to about 20 MHz. In lithotripsy, repetition rates of pulses are fairly slow, being measured in the hertz range, but the sharpness of the pulses generated give an effective pulse wavelength, or in this case, pulse rise time, with frequency content up to about 100 to about 300 MHz, or 0.1-0.3 gigahertz (GHz).

The frequency used in certain embodiments also may be influenced by the energy absorption characteristics of the sample or of the treatment vessel, for a particular frequency. To the extent that a particular frequency is better absorbed or preferentially absorbed by the sample, it may be desired. The energy may be delivered in the form of short pulses or as a continuous field for a defined length of time. The pulses may be bundled or regularly spaced.

A generally vertically oriented focused ultrasound beam may be generated in several ways. For example, a single-element piezoelectric transducer, such as those supplied by Sonic Concepts, Woodinville, Wash., that may be a 1.1 MHz focused single-element transducer, may have a spherical transmitting surface that is oriented such that the focal axis is vertical. Another embodiment uses a flat unfocused transducer and an acoustic lens to focus the beam. Still another embodiment uses a multi-element transducer such as an annular array in conjunction with focusing electronics to create the focused beam. The annular array potentially may reduce sonic sidelobes near the focal point by means of electronic apodizing, that is, by reducing the sonic energy intensity, either electronically or mechanically, at the periphery of the transducer. This result may be achieved mechanically by partially blocking the sound around the edges of a transducer or by reducing the power to the outside elements of a multi-element transducer. This reduces sidelobes near the energy focus, and may be useful to reduce heating of the vessel. Alternatively, an array of small transducers may be synchronized to create a converging beam. Still another embodiment combines an unfocused transducer with a focusing acoustic mirror to create the focused beam. This embodiment may be advantageous at lower frequencies when the wavelengths are large relative to the size of the transducer. The axis of the transducer of this embodiment may be horizontal and a shaped acoustic mirror used to reflect the acoustic energy vertically and focus the energy into a converging beam.

In certain embodiments, the focal zone may be small relative to the dimensions of the treatment vessel to avoid heating of the treatment vessel. In one embodiment, the focal zone has a radius of approximately 1 mm and the treatment vessel has a radius of at least about 5 mm. Heating of the treatment vessel may be reduced by minimizing acoustic sidelobes near the focal zone. Sidelobes are regions of high acoustic intensity around the focal point formed by constructive interference of consecutive wavefronts. The sidelobes may be reduced by apodizing the transducer either electronically, by operating the outer elements of a multi-element transducer at a lower power, or mechanically, by partially blocking the acoustic waves around the periphery of a single element transducer. Sidelobes may also be reduced by using short bursts, for example in the range of about 3 to about 5 cycles in the treatment protocol.

The transducer may be formed of a piezoelectric material, such as a piezoelectric ceramic. The ceramic may be fabricated as a “dome,” which tends to focus the energy. One application of such materials is in sound reproduction. However, as used herein, the frequency is generally much higher and the piezoelectric material would be typically overdriven, that is driven by a voltage beyond the linear region of mechanical response to voltage change, to sharpen the pulses. Typically, these domes have a longer focal length than that found in lithotriptic systems, for example, about 20 cm versus about 10 cm focal length. Ceramic domes may be damped to prevent ringing. The response is linear if not overdriven. The high-energy focus of one of these domes is typically cigar-shaped. At 1 MHz, the focal zone is about 6 cm long and about 2 cm in diameter for a 20 cm dome, or about 15 mm long and about 3 mm wide for a 10 cm dome. The peak positive pressure obtained from such systems is about 1 MPa (mega Pascal) to about 10 MPa pressure, or about 150 PSI (pounds per square inch) to about 1500 PSI, depending on the driving voltage.

The wavelength, or characteristic rise time multiplied by sound velocity for a shock wave, is in the same general size range as a cell, for example about 10 to about 40 micron. This effective wavelength may be varied by selection of the pulse time and amplitude, by the degree of focusing maintained through the interfaces between the source and the material to be treated, for example.

In certain embodiments, the focused ultrasound beam is oriented vertically in a water tank so that the sample may be placed at or near the free surface. The ultrasound beam creates shock waves at the focal point. In an embodiment to treat industry standard microplates which hold a plurality of cells and material in an array, a focal zone, defined as having an acoustic intensity within about 6 dB of the peak acoustic intensity, is formed around the geometric focal point. This focal zone has a diameter of approximately 2 mm and an axial length of about 6 mm.

Ceramic domes are adaptable for in vitro applications because of their small size. Also, systems utilizing ceramic domes may be produced at reasonable cost. They also facilitate scanning the sonic beam focus over a volume of liquid, by using microactuators which move a retaining platform to which the sample treatment vessel is attached.

Another source of focused pressure waves is an electromagnetic transducer and a parabolic concentrator, as is used in lithotripsy. The excitation tends to be more energetic, with similar or larger focal regions. Strong focal peak negative pressures of about −16 MPa have been observed. Peak negative pressures of this magnitude provide a source of cavitation bubbles in water, which may be desirable in an extraction process.

The examples described below use a commercial ultrasonic driver using a piezoelectric ceramic, which is stimulated by application of fluctuating voltages across its thickness to vibrate and so to produce acoustic waves. These may be of any of a range of frequencies, depending on the size and composition of the driver. Such drivers are used in lithotripsy, for example, as well as in acoustic speakers and in ultrasound diagnostic equipment, although without the control systems as described herein.

These commercially-available drivers have a single focus. Therefore, to treat an entire microplate with such a device, it is typically necessary to sequentially position or step each well at the focus of the driver. Because stirring time is brief, the stepping of a 96 well plate may be accomplished in approximately two minutes or less with simple automatic controls, as described below. It is contemplated that this time may be shortened.

It also is possible to make multi-focal drivers by making piezoelectric devices with more complex shapes. Modulators of the acoustic field attached to an existing piezoelectric driver may also produce multiple foci. These devices may be important for obtaining rapid throughput of microplates in a high density format, such as the 1534-well format.

One treatment protocol may include variable sonic waveforms combined with sample motion and positioning to achieve a desired effect. The sonic waveform of the transducer has many effects, including: sonic microstreaming in and near cells due to cavitation, that is flow induced by, for example, collapse of cavitation bubbles; shock waves due to nonlinear characteristics of the fluid bath; shock waves due to cavitation bubbles; thermal effects, which lead to heating of the sample, heating of the sample vessel, and/or convective heat transfer due to acoustic streaming; flow effects, causing deflection of sample material from the focal zone due to shear and sonic pressure, as well as mixing due to sonic streaming, that is flow induced by sonic pressure; and chemical effects.

The treatment protocol may be optimized to maximize energy transfer while minimizing thermal effects. The treatment protocol also may effectively mix the cells and material of the treatment vessel, in the case of a particulate sample suspended in a liquid. Energy transfer into the sample may be controlled by adjusting the parameters of the acoustic wave such as frequency, amplitude, and cycles per burst. Temperature rise in the sample may be controlled by limiting the duty cycle of the treatment and by optimizing heat transfer between the treatment vessel and the water bath. Heat transfer may be enhanced by making the treatment vessel with thin walls, of a relatively highly thermally conductive material, and/or by promoting forced convection by acoustic streaming in the treatment vessel and in the fluid bath in the proximity of the treatment vessel. Monitoring and control of temperature is discussed in more detail below.

For example, for a cellular disruption and insertion treatment, an example of an effective energy waveform is a high amplitude sine wave of about 1000 cycles followed by a dead time of about 9000 cycles, which is about a 10% duty cycle, at a frequency of about 1.1 MHz. The sine wave electrical input to the transducer typically results in a sine wave acoustic output from the transducer. As the focused sine waves converge at the focal point, they may become a series of shock waves due to the nonlinear acoustic properties of the water or other fluid in the bath. This protocol treats the material in the focal zone effectively during the “on” time. As the material is treated, it typically is expelled from the focal zone by acoustic shear and streaming. New material circulates into the focal zone during the “off” time. This protocol may be effective, for example, for inserting material into ground or particulate leaf tissue, while causing minimal temperature rise in the treatment vessel.

Further advantage in disruption and other processes may be gained by creating a high power “treat” interval alternating with a low power “mix” interval. More particularly, in this example, the “treat” interval uses a sine wave that has a treatment frequency, a treatment cycles-per-burst count, and a treatment peak-to-peak amplitude. The “mix” interval has a mix frequency, a mix cycles-per-burst count, and a lower mix peak-to-peak amplitude. Following each of the intervals is a dead time. Of course, these relationships are merely one example of many, where one interval is considered to be high power and one interval is considered to be low power, and these variables and others may be altered to produce more or less energetic situations. Additionally, the treat function or interval and the mix function or interval could emit from different or multiple transducers in the same apparatus, optionally emitting at different frequencies.

High power/low power interval treatments may allow multiple operations to be performed, such as altering permeability of components, such as cells, within the sample followed by subsequent mixing of the sample. The treat interval may maximize cavitation and bioeffects, while the mix interval may maximize mixing within the treatment vessel and/or generate minimal heat. Adding a longer, high power “super-mix” interval occasionally to stir up particles that are trapped around the periphery of the treatment vessel may provide further benefits. This “super-mix” interval generates additional heat, so it is programmed to treat infrequently during the process, for example, every few seconds. Additionally, dead times between the mix and treat intervals, during which time substantially no energy is emitted from the sonic energy source, may allow fresh material to circulate into the energy focal zone of the target.

As discussed below, moving the sample vessel during treatment relative to the source, so that the focal zone moves within the treatment vessel, may further enhance the process. For example, target motion through the focal zone may re-suspend material in the sample that may have clumped or become trapped around the periphery of the treatment vessel. A similar improvement may be achieved by traversing or “dithering” the treatment vessel relative to the focal zone. Dithering may become increasingly advantageous as the sample treatment vessel becomes significantly larger than the focal zone.

The waveform of focused sound waves may be a single shock wave pulse, a series of individual shock wave pulses, a series of shock wave bursts of several cycles each, or a continuous waveform. Incident waveforms may be focused directly by either a single element, such as a focused ceramic piezoelectric ultrasonic transducer, or by an array of elements with their paths converging to a focus. Alternatively, multiple foci may be produced to provide ultrasonic treatment to multiple treatment zones, vessels, or wells.

Reflected waveforms may be focused with a parabolic reflector, such as is used in an “electromagnetic” or spark-gap type shock-wave generator. Incident and reflected waveforms may be directed and focused with an ellipsoidal reflector such as is used in an electrohydraulic generator. Waveforms also may be channeled.

The waveform of the sound wave typically is selected for the particular material being treated. For example, to enhance cavitation, it may be desirable to increase the peak negative pressure following the peak positive pressure. For other applications, it may be desirable to reduce cavitation but maintain the peak positive pressure. This result may be achieved by performing the process in a pressurized chamber at a slight pressure above ambient. For example, if the waveform generated has a peak negative pressure of about 5 MPa, then the entire chamber may be pressurized to about 10 MPa to eliminate cavitation from occurring during the process. Liquid to be treated may be pressurized on a batch or a continuous basis.

A variety of methods of generating waves may be used. In lithotripsy, for example, “sharp” shock waves of high intensity and short duration are generated. Shock waves may be generated by any method that is applicable to a small scale. Such methods include spark discharges across a known gap; laser pulses impinging on an absorptive or reflective surface; piezoelectric pulses; electromagnetic shock waves; electrohydraulic shock waves created by electrical discharges in a liquid medium; and chemical explosives. In the case of explosives, micro-explosives in wells in a semiconductor-type chip may be fabricated in which the wells are individually addressable. Also, a magnetostrictive material may be exposed to a magnetic field, and it may expand and/or contract such that the material expansion/contraction creates sonic energy.

Continuous sinusoidal sound waves may be generated by any process that is appropriate for focusing on a small scale. For example, ceramic piezoelectric elements may be constructed into dome shapes to focus the sound wave into a point source. In addition, two or more shock waves may be combined from the same source, such as piezoelectric elements arranged in mosaic form, or from different sources, such as an electromagnetic source used in combination with a piezoelectric source, to provide a focused shock wave.

Typically, the shock wave is characterized by a rapid shock front with a positive peak pressure in the range of about 15 MPa, and a negative peak pressure in the range of about negative 5 MPa. This waveform is of about a few microseconds duration, such as about 5 microseconds. If the negative peak is greater than about 1 MPa, cavitation bubbles may form. Cavitation bubble formation also is dependent upon the surrounding medium. For example, glycerol is a cavitation inhibitive medium, whereas liquid water is a cavitation promotive medium. The collapse of cavitation bubbles forms “microjets” and turbulence that impinge on the surrounding material.

The waves are applied to the samples either directly, as for example, piezoelectric pulses, or via an intervening medium. This medium may be water or other fluid. An intervening medium also may be a solid, such as a material which is intrinsically solid or a frozen solution. Waves also may be applied through a container, such as a bottle, bag, box, jar, or vial.

For maximum control, and particularly for well-by-well mixing, a focused acoustic pulse is useful. When a pulse is emitted from a curved source with an elliptical profile, then the emitted sonic waves or pulses focus in a small region of maximum intensity. The location of the focus may be calculated or determined readily by experiment. The diameter of the focal zone may be of the same general size as or smaller than the diameter of the treatment vessel. Then, mixing energy may be provided to each well for a repeatable amount of time, providing uniform mixing of each sample.

In certain embodiments, the cells are not only moved into position relative to the transducer initially, but positioned during treatment to insure uniform treatment of the cells, where the cells are kept well suspended during treatment. As used herein, x and y axes define a plane that is substantially horizontal relative to ground and/or a base of an apparatus, while the z axis lies in a plane that is substantially vertical relative to the ground and/or the base of an apparatus and perpendicular to the x-y plane.

One positioning scheme is termed “dithering,” which entails slightly varying the position of the cells relative to the source which may occur by moving the sample through the focal zone in several ways. For example, but without limitation, the cells may be moved in a circle, or oval, or other arcuate path with a certain radius and moved a certain distance in certain increments or steps. These movements may vary between treatment cycles or during a particular treatment cycle and have several effects. First, dithering the cell position sweeps the focal zone through the volume of the sample treatment vessel or device, treating material that is not initially in the focal zone. In addition, varying the location of the sonic focus within the vessel tends to make treatment, and the resulting heating, more uniform within each sample.

Certain embodiments include drive electronics and devices for positioning of the sample(s). In one embodiment, the positioning sequence, optionally including dithering, and the treatment pulse train are pre-programmed, for example in a computer, and are executed automatically. The driver electronics and positioners may be linked through the control system to sensors so that there is “real time” feedback of sensor data to the control system during treatment in order to adjust the device(s) for positioning the sample and prevent localized heating or cavitation. The drive electronics may include a waveform generator matching network, an RF switch or relay, and a radio frequency (RF) amplifier, for safety shutdown.

The positioning system may include a three axis Cartesian positioning and motion control system to position the sample treatment vessel or an array of sample treatment vessels relative to the ultrasound transducer. The “x” and “y” axes of the Cartesian positioning system allow each sample in an array of samples, such as an industry standard microplate, to be brought into the focal zone for treatment. Alternative configurations may employ a combination of linear and rotary motion control elements to achieve the same capabilities as the three axis Cartesian system. Alternative positioning systems may be constructed of self-contained motor-driven linear or rotary motion elements mounted to each other and to a base plate to achieve two- or three-dimensional motion.

Stepper motors, such as those available from Eastern Air Devices, located in Dover, N.H., drive linear motion elements through lead screws to position the sample. The stepper motors are driven and controlled by means of LabVIEW software controlling a ValueMotion stepper motor control board available from National Instruments, located in Austin, Tex. The output signals from the control board are amplified by a nuDrive multi-axis power amplifier interface, also available from National Instruments, to drive the stepper motors.

The computer controlled positioning system may be programmed to sequentially move any defined array of multiple samples into alignment with the focal zone of the ultrasound transducer. If temperature rise during treatment is an issue, the samples in a multi-sample array may be partially treated and allowed to cool as the positioning system processes the other samples. This may be repeated until all the samples have been treated fully.

The positioning system also may move the sample treatment vessel relative to the focal point during treatment to enhance the treatment or to treat a sample that is large relative to the focal zone. By sweeping the sample slowly in a circular or other motion during treatment, clumps of material around the periphery of the treatment vessel may be broken up advantageously. In addition, x-y dithering may prevent a “bubble shield” from forming and blocking cavitation in the sample treatment vessel. The x-y dithering may also enhance treatment of sample suspensions that have a high viscosity or become more viscous during treatment and do not mix well. The sample position may also be dithered vertically in the z-axis. This may be advantageous in a deep treatment vessel where the depth is substantially larger than the axial dimension of the focal zone, in order to treat the entire contents of the treatment vessel or to re-suspend larger sample fragments which have sunk to the bottom of the vessel. Dithering in all three dimensions may also be employed.

For a relatively flat sample, such as whole leaf tissue, a histological sample, or thin-section specimen, where the area of the sample is large relative to the cross-sectional area of the focal zone, the x-y positioning system may cause the focal zone to traverse the sample in order to treat the entire surface of the sample. This procedure may be combined with optical analysis or other sensors to determine the extent of the treatment to each portion of the sample that is brought into the focal zone.

In certain embodiments, the sample or array of samples may be moved relative to the transducer and the other parts of the apparatus. In alternative embodiments the transducer is moved while the sample holder remains fixed, relative to the other parts of the apparatus. As an alternative, movement along two of the axes, for example, x and y, may be assigned to the sample holder and movement along the third axis, z in this case, may be assigned to the transducer.

The three axis positioning system enables automated energy focus adjustment in the z-axis when used in conjunction with a sensor for measuring the ultrasound intensity. In one embodiment, a needle hydrophone may be mounted in a fixture on the sample positioning system. The hydrophone may be traversed in three dimensions through the focal region to record the acoustic intensity as a function of position in order to map out the focal zone. In another embodiment, a number of positions on a sheet of aluminum foil held in the sample holder may be treated in a sequence of z-axis settings. The foil may then be examined to determine the spot size of the damage at each position. The diameter of the spot corresponds generally to the diameter of the focal zone at that z-axis setting. Other, fully automated embodiments of a focusing system may also be constructed.

The three axis positioning system also allows the apparatus to be integrated into a larger laboratory automation scheme. A positioning system with an extended work envelope may transfer microplates or other sample vessels into and out of the apparatus. This allows the apparatus to interact automatically with upstream and downstream processes.

Optical or video detection and analysis may be employed to optimize treatment of the sample. For example, in a suspension of biological tissue, the viscosity of the mixture may increase during treatment due to the diminution of the particles by the treatment and/or by the liberation of macromolecules into the solution. Video analysis of the sample during treatment allows an automated assessment of the mixing caused by the treatment protocol. The protocol may be modified during the treatment to promote greater mixing as a result of this assessment. The video data may be acquired and analyzed by the computer control system that is controlling the treatment process. Other optical measurements such as spectral excitation, absorption, fluorescence, emission, and spectral analysis also may be used to monitor treatment of the sample. A laser beam, for example, may be used for alignment and to indicate current sample position.

Heating of individual wells may be determined by an infrared temperature-sensing probe, collimated so as to view only the well being treated with the ultrasonic energy. For example, an infrared thermal measuring device may be directed at the top unwetted side of the treatment vessel. This provides a non-contact means of analysis that is not readily achievable in conventional ultrasound treatment configurations. The thermal information may be recorded as a thermal record of the sample temperature profile during treatment.

Active temperature monitoring may be used as a feedback mechanism to modify the treatment protocol during the treatment process to keep the sample temperature within specified limits. For example, an infrared sensor directed at the sample treatment vessel may input temperature readings to the computer. The computer, in accordance with a controlling program, may produce output directed to the circuit enabling the ultrasonic transducer, which in turn may reduce the high power treatment intervals and increase the low power mixing intervals, for example, if the sample temperature is nearing a specified maximum temperature.

A variety of methods may be employed to detect cavitation. For example, sonic emissions, optical scattering, high-speed photography, mechanical damage, and sonochemicals may be used. As described above for monitoring temperature, information from cavitation detection may be used by the system to produce an output that selectively controls exposure of a sample to sonic energy in response to the information. Each of these methods to monitor cavitation are described more fully below.

Bubbles are effective scatterers of ultrasound. The pulsation mode of a bubble is referred to as monopole source, which is an effective acoustic source. For small, generally linear oscillations, the bubble simply scatters the incident acoustic pulse. However, as the response becomes more nonlinear, it also starts to emit signals at higher harmonics. When driven harder, the bubbles start to generate sub-harmonics as well. Eventually as the response becomes aperiodic or chaotic, the scattered field tends towards white noise. In the scenario where inertial collapses occur, short acoustic pressure pulses are emitted. An acoustic transducer may be configured to detect these emissions. There is a detectable correlation between the onset of the emissions and cell disruption.

Bubbles also scatter light. When bubbles are present, light is scattered. Light may normally be introduced into the system using fiber optic light sources so that cavitation may be detected in real-time, and therefore may be controlled by electronic and computer systems.

Bubbles may be photographed. This method typically requires high-speed cameras and high intensity lighting, because the bubbles respond on the time frame of the acoustics. It also requires good optical access to the sample under study. This method may give detailed and accurate data and may be a consideration when designing systems for implementing the methods herein. Stroboscopic systems, which take images far less frequently, may often give similar qualitative performance more cheaply and easily than high-speed photography.

Cavitation is known to create damage to mechanical systems. Pitting of metal foils is a particularly common effect, and detection method. There is a correlation between the cavitation needed to pit foils and to disrupt cells.

A number of chemicals are known to be produced in response to cavitation. The yield of these chemicals may be used as a measure of cavitational activity. A common technique is to monitor light generation from chemicals, such as luminol, that generate light when exposed to cavitation. Sonochemical yield usually may not be done during cell experiments but may be done independently under identical conditions, and thereby, provide a calibrated standard.

The introduction of a material into the interior of a cell may require that the temperature be managed and controlled during processing. For example, many biological samples should not be heated above 4° C. during treatment. The ultrasound treatment protocol influences the sample temperature in several ways: the sample absorbs sonic energy and converts it to heat; the sample treatment vessel absorbs sonic energy and converts it to heat which, in turn, may heat the sample; and sonic streaming develops within the sample treatment vessel and the water bath, forcing convective heat transfer between the sample treatment vessel and the water bath. In the case of a relatively cool water bath, this cools the sample.

The sonic waves or pulses may be used to regulate the temperature of the solutions in the treatment vessel. At low power, the sonic energy produces a slow stirring without marked heating. Although energy is absorbed to induce the stirring, heat is lost rapidly through the sides of the treatment vessel, resulting in a negligible equilibrium temperature increase in the sample. At higher energies, more energy is absorbed, and the temperature rises. The degree of rise per unit energy input may be influenced and/or controlled by several characteristics, including the degree of heat absorption by the sample or the treatment vessel and the rate of heat transfer from the treatment vessel to the surroundings. Additionally, the treatment protocol may alternate a high-powered treatment interval, in which the desired effects are obtained, with a low power mixing interval, in which sonic streaming and convection are achieved without significant heat generation. This convection may be used to promote efficient heat exchange or cooling.

The thermal information may also be used to modify or control the treatment to maintain the sample temperature rise below a maximum allowable value. The treatment may be interrupted to allow the sample to cool down. In certain embodiments, the output of the thermal measurement device or system is entered into the computer control system for recording, display on a control console, and/or control of exposure of the sample to sonic energy through a feedback loop, for example, by altering the duty cycle.

Temperature rise during ultrasonic continuous wave exposure may be controlled by refrigeration of a liquid or other sample before, during, or after passage through a zone of sonic energy, if processing in a continuous, flow-through mode. In generally stationary discrete sample processing modes, a sample may be cooled by air, by contact with a liquid bath, or a combination of both air and liquid. The temperature is rapidly equilibrated within the vessel by the stirring action induced by the sonic waves. As a result, and especially in small vessels or other small fluid samples, the rate of temperature increase and subsequent cooling may be very rapid. The rate of delivery of sonic energy to the material may also be controlled, although that may lengthen processing time.

Liquids within the sample may be provided at any temperature compatible with the process. The liquid may be frozen or partially frozen for processing. For example, when biological material is subjected to subzero temperatures below about −5° C. most, but not all, of the water is in the solid phase. However, in certain biological tissues, micro-domains of liquid water still remain for several reasons, such as natural “antifreeze” molecules or regions of higher salt concentration. Therefore, sample temperature may be varied during the procedure. A temperature is selected at which micro-domains of liquid water are able to form shock wave induced cavitation due to bubble formation and collapse, resulting in shear stresses that impinge on surrounding tissues. Indeed, gradually altering the sample temperature may be desirable, as it provides focused domains of liquid water for collection of sonic energy for impingement on the surrounding material.

Treatment baths may be relatively simple, and may include a water bath or other fluid bath that is employed to conduct the sonic waves from the transducer to the sample treatment vessel, where the liquid is temperature controlled. In certain embodiments, the entire bath is maintained at a specific temperature by means of an external heater or chiller, such as a Neslab RTE-210 chiller available from Neslab Instruments, Inc., located in Newington, N.H., and heat exchanger coils immersed in the bath. The sides and bottom of the tank containing the bath may have sufficient insulating properties to allow the bath to be maintained substantially uniformly at a specific temperature. Another embodiment employs an inner tray or sample tank made of an insulating material such as rigid polystyrene foam which is set within a larger water bath in a transducer tank. An inner tray has heat-exchanger tubes or other heating or cooling devices within it to allow a fluid such as ethylene glycol or propylene glycol in the inner tray to be heated or cooled beyond what may be practical for the fluid such as water in the outer bath in the transducer tank. The inner tray has an acoustic window in the bottom. The acoustic window is made of a thin film material having low acoustic absorption and an acoustic impedance similar to water. This inner tray is arranged so that the acoustic window is aligned with a transducer which is outside the tray, supported with a support in the water. A sample is located within a microtiter plate or other sample treatment vessel, within the tray and is subjected to the thermal influence of the inner treatment bath. The treatment vessel may be movable relative to the transducer with a positioning system. Also, sonic energy focuses on the sample through the acoustic window. This arrangement permits the use of separate fluids and substantially independent control of the temperature of the inner and outer treatment baths. The smaller volume of the inner tray facilitates the use of antifreeze mixtures, such as a mixture of propylene glycol and water, at temperatures below the freezing temperature of water. This, in turn, allows the samples to be processed and treated at temperatures below the freezing temperature of water. This embodiment is beneficial for treatment applications requiring that the sample materials be maintained at temperatures near or below the freezing point of water. It allows for the containment of treatment bath fluids, such as antifreeze solutions, that may not be compatible with the transducer and other system components. It also allows the transducer to be maintained at a different temperature than the samples.

Sample temperature may be required to remain within a given temperature range during a treatment procedure. Temperature may be monitored remotely by, for example, an infra-red sensor. Temperature probes such as thermocouples may not be particularly well suited for all applications because the sound beam may interact with the thermocouple and generate an artificially high temperature in the vicinity of the probe. Temperature may be monitored by the same computer that controls sonic waveform. The control responds to an error signal which is the difference between the measured actual temperature of the sample and the target temperature of the sample. The control algorithm may be as a hysteritic bang-bang controller, such as those in kitchen stoves, where, as an output of the control system, the sonic energy is turned off when the actual temperature exceeds a first target temperature and turned on when the actual temperature falls below a second target temperature that is lower than the first target temperature. More complicated controllers may be implemented. For example, rather than simply turning the sonic signal on and off, the sonic signal could continuously be modulated proportionally to the error signal, for example, by varying the amplitude or the duty cycle, to provide finer temperature regulation.

In the application of a bang-bang control algorithm for a multiple sample format, once a maximum temperature value has been exceeded and the sonic energy is turned off for a particular sample, an alternative to waiting for the sample to cool below a selected temperature before turning the sonic energy on again, is to move on to the next sample. More particularly, some of the samples may be at least partially treated with sonic energy, in a sequence, and then, the system may return to the previously partially treated samples to take a sensor reading to determine if the samples have cooled below the selected temperature and to reinitiate treatment if they have. This procedure treats the samples in an efficient manner and reduces the total treatment time for treating multiple samples. Another alternative is to switch to a predefined “cooling” waveform which promotes convection without adding significant heat to a particular sample, rather than moving on to the next sample and returning to the first sample at a later time.

If uniformity of temperature throughout the sample is important, then control techniques may be used to ensure a uniform temperature distribution. An array of infra-red sensors may be used to determine the distribution of the temperature inside the sample. If areas of increased temperature relative to the rest of the sample appear, then the transducer may be switched from high power “treatment” mode to low power “mixing” mode. In the low power “mixing” mode, the sample is sonically stirred until the sample is substantially uniform in temperature. Once temperature uniformity is achieved, the high power “treatment” mode is reinitiated. A control system may monitor temperature and responsively turn the various modes on or off. When controlled by a computer, the intervals during which these modes are used may be very short, for example fractions of a second, thereby not significantly prolonging treatment times. Stepping times between wells, or other sample containers, may also be less than a second with suitable design.

In some applications, it may be desirable to treat the sample with as much energy as possible without causing cavitation. This result may be achieved by suppressing cavitation. Cavitation may be suppressed by pressurizing the treatment vessel above ambient, often known as “overpressure,” to the point at which no negative pressure develops during the rarefaction phase of the sonic wave. This suppression of cavitation is beneficial in applications such as cell transformation where the desired effect is to open cellular membranes while maintaining viable cells. In other applications it may be desirable to enhance cavitation. In these applications, a “negative” overpressure or vacuum may applied to the region of the focal zone.

The control of cavitation in the sample also may be important during sonic treatment processes. In some scenarios, the presence of small amounts of cavitation may be desirable to enhance biochemical processes; however, when large numbers of cavitation bubbles exist they may scatter sound before it reaches the target, effectively shielding the sample.

Cavitation may be detected by a variety of methods, including sonic and optical methods. An example of sonic detection is a passive cavitation detector (PCD) which includes an external transducer that detects sonic emissions from cavitation bubbles. The signal from the PCD may be filtered, for example, using a peak detector followed by a low pass filter, and then input to a controlling computer as a measure of cavitation activity. The sonic signal could be adjusted in ways similar to those described in the temperature control example to maintain cavitation activity at a desired level.

Increased ambient pressure is one technique for controlling cavitation. Overpressure tends to remove cavitation nuclei. Motes in the fluid are strongly affected by overpressure and so cavitation in free-fluid is often dramatically reduced, even by the addition of one atmosphere of overpressure. Nucleation sites on container walls tend to be more resistant to overpressure. However, the cavitation tends to be restricted to these sites and any gas bubbles that float free into the free-fluid are quickly dissolved. Therefore cells in the bulk fluid are typically unaffected by cavitation sites restricted to the container walls. Overpressure may be applied to the treatment vessel, the array of treatment vessels, the treatment bath and tank, or to the entire apparatus to achieve a higher than atmospheric pressure in the region of the focal zone.

Reducing the gas content of the fluid tends to reduce cavitation, again by reducing cavitation nuclei and making it harder to initiate cavitation. Another method of controlling cavitation or the effects of cavitation is to control the gasses that are dissolved in the sample fluid. For instance, cavitation causes less mechanical damage in fluid saturated with helium gas than in fluid saturated with argon gas. Cleaner fluids tend to be harder to cavitate.

Certain fluids are much harder to cavitate. Castor oil and mineral oil are nearly cavitation free. Two possible reasons are that the fluids are of a nature that they tend to fill in cracks, and that their viscosity also makes them more resistant to cavitation. The fluids, however, are not particularly compatible with cell preparations.

The cavitation field responds to the acoustic driving pulse. It is possible to control the cavitation response, to some extent, by controlling the driving sonic pressure. Cavitation may also be reduced or eliminated by reducing the number of cycles in each burst of sonic energy. The cavitation bubbles grow over several cycles then collapse creating cavitation effects. By limiting the number of cycles in each burst, bubble growth and collapse may be substantially avoided.

Treatment vessels are sized and shaped as appropriate for the material to be treated. They may be any of a variety of shapes. For example, treatment vessels may have vertical walls, may have a conical shape, or may have a curved shape, respectively. Certain treatment vessel prior to treatment with sonic energy, have an upper member and a lower member which together form an interior region that contains the material to be treated. In certain embodiments, the ultrasound transducer projects a focused ultrasound beam upwards. The ultrasound beam penetrates the lower member of the treatment vessel to act upon the contents of the treatment vessel. The upper member serves to contain the contents of the vessel.

The lower member of the treatment vessel is configured to transmit the maximum amount of ultrasound energy to the contents of the vessel, minimize the absorption of ultrasound energy within the walls of the vessel, and maximize heat transfer between the contents of the treatment vessel and, for example, an external water bath. In certain embodiment of the pre-treatment assembly, the treatment vessel is thermoformed from a thin film in a hemispherical shape. The film should have an acoustic impedance similar to that of water and low acoustic absorption. One desired material is low density polyethylene. Alternative materials include polypropylene, polystyrene, poly(ethylene teraphthalate) (“PET”), and other rigid and flexible polymers. The film may be a laminate to facilitate thermal bonding, for example using heat sealing. Thicker, more rigid materials may also be employed. Available multi-well plates in industry standard formats such as 96 well and 24 well formats may be employed with or without modification. Industry standard thick-wall, multi-well plates with thin film bottoms may also be employed. These may work particularly well where the size of the focal zone of the ultrasound beam is smaller than a well. In this case, little energy is absorbed by the sides of the treatment vessel and, as a result, relatively little energy is converted to heat.

The upper member of the treatment vessel contains the contents in the vessel during treatment and may act also as an environmental seal. The upper member of the treatment vessel may be flat or domed to enclose the interior of the treatment vessel. The upper member of the treatment vessel may be made of a rigid or flexible material. It may be desirable that the material have low sonic absorption and good heat transfer properties. In certain embodiments of the pre-treatment assembly, the upper member of the treatment vessel is a thin film that may be bonded to the lower member, and the lower or upper member may be easily rupturable for post-treatment transfer of the treated material.

The upper and lower members of the treatment vessel may be joined together by thermal bonding, adhesive bonding, or external clamping. Such joining of the upper and lower members may serve to seal the contents of the vessel from contaminants in the external environment and, in an array of vessels, prevent cross-contamination between vessels. If the bond is to be achieved by thermal bonding, the upper and lower members of the treatment vessels may be made of film laminates having heat bondable outer layers and heat resistant inner layers.

The treatment vessel may be configured as a single unit, as a multiplicity of vessels in an array, or as a single unit with various compartments. The upper and lower members of the vessel or array of vessels may be used once or repeatedly. There also may be a separate frame or structure that supports and/or stiffens the upper and lower members of the vessel(s). This frame or structure may be integral with the vessels or may be a separate member. An array of treatment vessels may be configured to match industry standard multi-well plates. In one embodiment, the treatment vessel is configured in an array that matches standard 96 well or 24 well multi-well plates. The frame or supporting structure holding the array of treatment vessels may have the same configuration and dimensions as standard multi-well plates.

A treatment vessel may include a funnel to facilitate transfer of the contents from the treatment vessel to a separate vessel after treatment. The funnel may have a conical shape and include an opening at the narrow end. The funnel may be rigid, relative to the upper and lower members of the treatment vessel. The large end of the funnel is proximate the upper member of the treatment vessel and aligned with the treatment vessel. The volume of the funnel may be marginally less than the volume of the treatment vessel.

One process of transferring the contents of the treatment vessel to another post-treatment vessel includes the following steps. The upper member of the treatment vessel may be pierced with a sharp instrument or ruptured when a vacuum is applied. To facilitate rupture, the member may be manufactured from a thin fragile material or made weak by etching a feature into the surface. Then, the treatment vessel is inverted over the post-treatment vessel in a vacuum fixture. A filter may be placed between the treatment vessel and the post-treatment vessel to separate solids from the liquid that is removed from the treatment vessel. Alternatively, the filter may be incorporated into the outlet of the funnel. This arrangement of treatment vessel and funnel may be configured as a single unit or as an array of units. This array may match an industry standard. The treatment vessel may form a vacuum seal with a vacuum fixture such that a pressure differential may form between the sample in the treatment vessel and the supplied vacuum. Once the vacuum is applied to the fixture, the pressure differential across the upper member may cause the upper member of the treatment vessel to rupture and cause the lower member to collapse into the funnel. The lower member may have sufficient strength so that it does not rupture where it bridges the opening in the small end of the funnel. The pressure differential may cause the solid contents of the treatment vessel to be squeezed between the flexible lower member of the treatment vessel and the relatively rigid funnel. This causes fluid to be expelled from the solid materials and collected in the post-treatment vessel.

In certain other embodiments, a treatment vessel may be an ampoule, vial, pouch, bag, or envelope. These and other treatment vessels may be formed from such materials as polyethylene, polypropylene, poly(ethylene teraphthalate) (PET), polystyrene, acetal, silicone, polyvinyl chloride (PVC), phenolic, glasses and other inorganic materials, metals such as aluminum and magnesium, and laminates such as polyethylene/aluminum and polyethylene/polyester. Also, certain embodiments of a treatment vessel may be made by vacuum forming, injection molding, casting, and other thermal and non-thermal processes. In embodiments where samples flow through the sonic energy, capillary tubes, etched channels, and conduits may be the sample holder during treatment as the sample flows through a structure. Additionally, free-falling drops, streams, non-moving free volumes, such as those in gravity less than one g, or a layer in a density gradient may be treated directly.

Certain embodiments provide kits for conveniently and effectively implementing the methods described herein. Such kits comprise any subject composition, and a means for facilitating compliance with the methods herein. Such kits provide a convenient and effective means for assuring that the subject to be treated takes the appropriate active in the correct dosage in the correct manner. The compliance means of such kits includes any means which facilitates administering the actives according to a method herein. Such compliance means include instructions, packaging, and dispensing means, and combinations thereof. Kit components may be packaged for either manual or partially or wholly automated practice of the foregoing methods. In other embodiments involving kits, a kit is contemplated including compositions of the embodiments as described herein, and optionally instructions for their use.

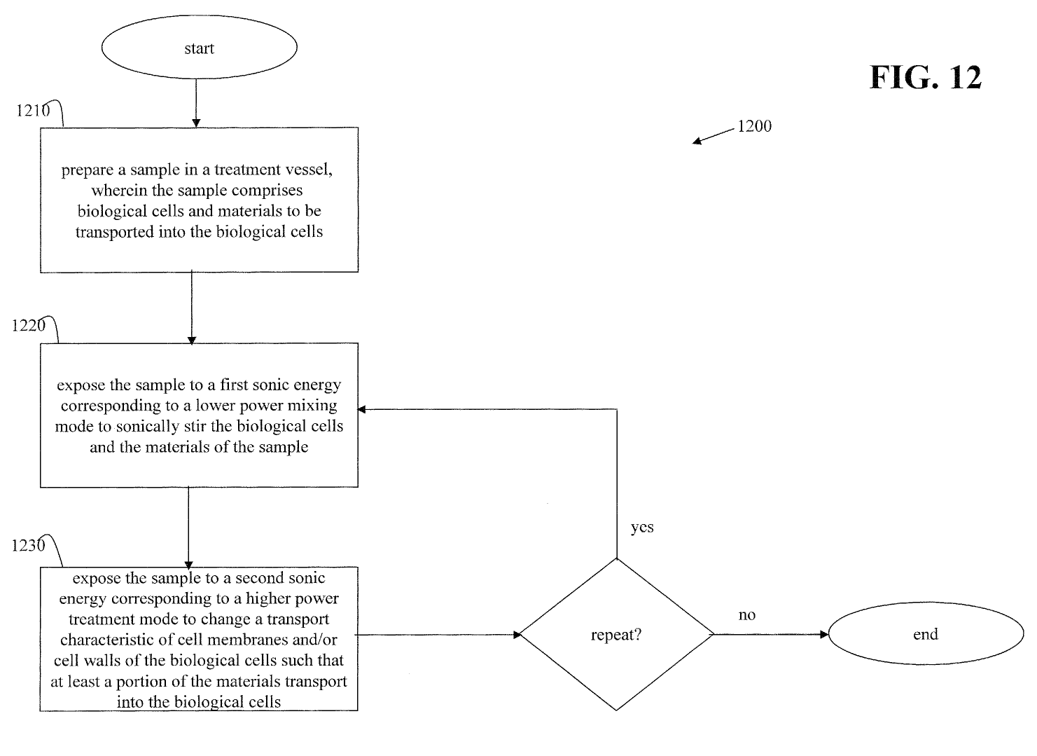

FIG. 12 is a flowchart of a third embodiment of a method 1200 of introducing materials into biological cells using sonic energy. In step 1210, a sample comprising biological cells and materials to be transported into the biological cells is prepared in a treatment vessel. In step 1220, the sample is exposed to a first sonic energy corresponding to a lower power mixing mode to sonically stir the biological cells and the materials of the sample. In step 1230, the sample is exposed to a second sonic energy corresponding to a higher power treatment mode to change a transport characteristic of cell membranes and/or cell walls of the biological cells such that at least a portion of the materials transport into the biological cells. Steps 1220 and 1230 may be repeated, at least once, to further mix the sample and transport more materials into the biological cells. In accordance with an embodiment, the lower power mixing mode and the higher power treatment mode are inhibited from being performed simultaneously.

For example, the lower power mixing mode comprises a first sonic waveform having a first frequency, a first cycles-per-burst count, and a first peak-to-peak amplitude. The higher power treatment mode comprises a second sonic waveform having a second frequency, a second cycles-per-burst count, and a second peak-to-peak amplitude. The sample may occasionally and/or periodically be further exposed to a third sonic energy corresponding to a higher power super-mix mode to sonically stir any of the biological cells and the materials of the sample that are trapped around a periphery of the treatment vessel.

FIG. 13 is a flowchart of an embodiment of a method 1300 of introducing materials into viable organisms using sonic energy. In step 1310, a sample is prepared in a treatment vessel, wherein the sample comprises viable organisms, a viable organism medium, and transportable materials. The viable organisms may be of a substantially singular type such as, for example, adult Hydra. The viable organism medium may comprise a Hydra medium. Alternatively, the viable organisms may comprise various different types. Similarly, the materials to be transported may be of a substantially singular type or comprise a variety of types. In step 1320, the sample is exposed to a pre-defined sonic energy waveform such that at least a portion of the transportable materials are transported into cells of the viable organisms via cell membranes and/or cell walls of the cells when a transport characteristic of the cells is changed in response to the exposing sonic energy waveform.

In summary, certain embodiments of methods have been described herein for controllably disrupting cell membranes and/or cell walls in biological cells using sonic energy, to introduce materials into the biological cells using sonic energy, and for correlating sonic energy exposure to transport efficiency into biological cells. Such methods facilitate the controlled introduction of various material types into various types of biological cells for various purposes.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. A method of transporting materials into biological cells ex-vivo using sonic energy, said method comprising:
extracting a sample of viable biological cells from a human subject;
preparing a mix comprising said sample of viable biological cells and materials to be transported;
exposing said mix to sonic energy of a pre-determined target energy level to isothermally change a transport characteristic of cell membranes and/or cell walls of said biological cells such that at least a portion of said materials transport into at least a portion of said biological cells and such that at least a portion of said biological cells remain viable;
terminating said exposing after a pre-determined exposure time such that said transported materials become trapped within said biological cells; and
re-introducing said viable biological cells into said human subject.

2. The method of claim 1 wherein said viable biological cells are of a substantially singular type.

3. The method of claim 1 wherein said materials are of a substantially singular type.

4. The method of claim 1 wherein said materials include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

5. The method of claim 1 wherein parameters of said exposing sonic energy include an ultrasonic frequency, a duty cycle, a number of cycles per burst, a peak amplitude, and an exposing time duration.

6. The method of claim 1 wherein parameters of said exposing sonic energy comprise about a 1% duty cycle, at about 10 to 50 cycles per burst, at about 100 to 300 mV, for about 5 to 10 seconds.

7. A method of introducing material into biological cells using sonic energy, said method comprising:
(a) preparing a mix comprising biological cells and materials to be transported into said biological cells;
(b) selecting a sonic energy waveform corresponding to a first set of sonic energy parameters previously associated with said biological cells and said materials based on transportation characteristics of said materials into said biological cells;
(c) exposing said mix to sonic energy, according to said selected waveform having said first set of sonic energy parameters, to change a transport characteristic of cell membranes and/or cell walls of said biological cells such that at least a portion of said materials are transported through said cell membranes and/or cell walls into at least a portion of said biological cells without substantially damaging said biological cells; and
(d) terminating said exposing to trap said transported materials within said biological cells.

8. The method of claim 7 wherein said biological cells are of a substantially singular type.

9. The method of claim 7 wherein said materials are of a substantially singular type.

10. The method of claim 7 wherein said sonic energy parameters include an ultrasonic frequency, a duty cycle, a number of cycles per burst, a peak amplitude, and an exposing time duration.

11. The method of claim 7 wherein said sonic energy parameters include about a 1% duty cycle, at about 10 to 50 cycles per burst, at about 100 to 300 mV, for about 5 to 10 seconds.

12. The method of claim 7 wherein said mix is formed in-vitro.

13. The method of claim 7 wherein said mix is formed ex-vivo.

14. A method of introducing materials into biological cells using sonic energy, said method comprising:
preparing a sample, wherein said sample comprises biological cells and materials to be transported into said biological cells; and
exposing said sample to sonic energy wherein said sonic energy is supplied at about a 1% duty cycle, at about 10 to 50 cycles per burst, at about 100 to about 300 mV, and for about 5 to about 10 seconds.

15. The method of claim 14 wherein said biological cells are of a substantially singular type.

16. The method of claim 14 wherein said materials are of a substantially singular type.

17. The method of claim 14 wherein said materials include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

18. A method of correlating sonic energy exposure to transport efficiency in biological cells, said method comprising:
(a) exposing a solution to a level of sonic energy for a period of time, where said solution comprises biological cells and materials to be transported into said biological cells;
(b) examining said biological cells, after said exposing, for transport of said materials into said cells and for cell damage;
(c) determining and recording an amount of transported materials at said level of sonic energy for said period of time in response to said examining;
(d) increasing said level of sonic energy and/or said period of time and repeating steps (a) through (d) if no cell damage was observed in step (b); and
(e) determining the sonic energy and/or period of time for substantially optimized transport efficiency relating to the type of biological cells and type of materials to be transported into said type of biological cells from step (d).

19. The method of claim 18 wherein said biological cells are of a substantially singular type.

20. The method of claim 18 wherein said materials are of a substantially singular type.

21. The method of claim 18 wherein said exposing is accomplished substantially isothermally.

22. The method of claim 18 wherein said materials include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

23. A method of introducing materials into biological cells using sonic energy, said method comprising:
(a) preparing a sample in a treatment vessel, wherein said sample comprises biological cells and materials to be transported into said biological cells;
(b) exposing said sample to a first sonic energy corresponding to a lower power mixing mode to sonically stir said biological cells and said materials of said sample; and
(c) exposing said sample to a second sonic energy corresponding to a higher power treatment mode to change a transport characteristic of cell membranes and/or cell walls of said biological cells such that at least a portion of said materials transport into said biological cells.

24. The method of claim 23 wherein said biological cells are of a substantially singular type.

25. The method of claim 23 wherein said materials are of a substantially singular type.

26. The method of claim 23 further comprising repeating steps (b) and (c) at least once.

27. The method of claim 23 wherein said lower power mixing mode includes a first sonic waveform having a first frequency, a first cycles-per-burst count, and a first peak-to-peak amplitude.

28. The method of claim 23 wherein said higher power treatment mode includes a second sonic waveform having a second frequency, a second cycles-per-burst count, and a second peak-to-peak amplitude.

29. The method of claim 26 further comprising exposing said sample to a third sonic energy corresponding to a higher power super-mix mode to sonically stir any of said biological cells and said materials of said sample that are trapped around a periphery of said treatment vessel.

30. The method of claim 23 wherein said materials include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

31. A method of introducing materials into viable organisms using sonic energy, said method comprising:
preparing a sample in a treatment vessel, wherein said sample comprises viable organisms, a viable organism medium, and transportable materials; and
exposing said sample to a pre-defined sonic energy waveform such that at least a portion of said transportable materials are transported into cells of said viable organisms via cell membranes and/or cell walls of said cells when a transport characteristic of said cells is changed by said exposing sonic energy waveform.

32. The method of claim 31 wherein said viable organisms are of a substantially singular type.

33. The method of claim 31 wherein said materials are of a substantially singular type.

34. The method of claim 31 wherein said pre-defined sonic energy waveform is defined by parameters including about a 1% duty cycle, at about 10 to 50 cycles per burst, at about 250 to 300 mV, for about 5 to 10 seconds.

35. The method of claim 31 wherein said transportable materials include at least one of chemicals, inorganic molecules, organic molecules, monomers, nucleic acids, amino acids, polymers, nucleic acid chains, proteins, viruses, plasmids, vectors, DNA, RNA, other nucleic acid constructs, nucleic acid monomers, monomers including saccharides, polymers including polysaccharides, amino acid chains, enzymes, cofactors, and visualization reagents such as fluorescent probes.

36. The method of claim 31 wherein said viable organisms comprise adult Hydra and said viable organism medium comprises a Hydra medium.
